(19) United States
(12) Patent Application Publication
(10) Pub. No.: US 2005/0234351 A1
(43) Pub. Date: Oct. 20, 2005

(54) Body condition measuring device

(75) Inventors: Katsuyoshi Nishii, Okazaki-city (JP); Teiyuu Kimura, Nogoya-city (JP); Kazuhiro Sakai, Anjo-city (JP); Fumiya Nagai, Anjo-city (JP); Kazuya Inokawa, Obu-city (JP)

Correspondence Address: POSZ LAW GROUP, PLC, 12040 SOUTH LAKES DRIVE, SUITE 101, RESTON, VA, 20191, US

(73) Assignee: DENSO CORPORATION

(21) Appl. No.: 11/106,518

(22) Filed: Apr. 15, 2005

(30) Foreign Application Priority Data
Apr. 16, 2004 (JP) 2004-121747
Nov. 22, 2004 (JP) 2004-337678

(51) Int. Cl. A61B 5/02

(52) U.S. Cl. 600/503

(57) ABSTRACT

A pulse wave sensor main unit is provided with a pair of band anchoring portions that are positioned in the direction of the circumference of the wrist and through which the band is passed. Strip-shaped band insertion holes are formed in the band anchoring portions. The band is an elastic long strip-shaped body. The band is formed of material such as rubber that is flexibly deformed when external force is applied and is restored to its original shape by its own elasticity when external force is removed. The band is so set that its thickness is larger than the dimension of the shorter sides of the band insertion holes and its width is larger than the dimension of the longer sides of the band insertion holes.

### CROSS REFERENCE TO RELATED APPLICATION

This application is based on and incorporates herein by reference Japanese Patent Application No. 2004-121747 filed on Apr. 16, 2004 and Japanese Patent Application No. 2004-337678 filed on Nov. 22, 2004.

### BACKGROUND OF THE INVENTION

1. Field of the Invention

The present invention relates to a body condition measuring device that is attached around, for example, a wrist of a human body and is capable of detecting pulse waves or other information of the human body.

2. Description of Related Art

Various pulse wave sensors have been conventionally used to detect the conditions of humans through pulse waves.

For example, JP-2001-276001A proposes a pulse wave sensor comprising a wrist watch-type sensor main unit and a band. In this pulse wave sensor, the band is provided with a plurality of adjustment holes and a tongue. The tongue is inserted into the adjustment holes to adjust a pressing force for pressing the pulse wave sensor against the wrist when the pulse wave sensor is attached to the wrist. In the pulse wave sensors, in general, a measurement error is prone to be produced when displacement of the pulse wave sensor occurs during measurement. To prevent the displacement during the measurement, the above-mentioned pulse wave sensor is constructed as follows. That is, trapezoidal protruded elastic bodies are disposed on the inner side (the side opposite the sensor main unit) of the band of the pulse wave sensor.

For the purposes of preventing the displacement and the like, the technology disclosed in JP-2003-220041A takes the following measures. That is, in order to hold the pulse wave sensor with constant pressing force, part of the band of the pulse wave sensor is formed of an elastic body, and the elastic body has indicative portions, each of which indicates the corresponding strength of the elastic body.

However, the technology disclosed in JP-2001-276001A poses the following disadvantage. That is, when a user wears the pulse wave sensor, he/she gets annoyed with the protrusions of elastic body. The technology also poses another disadvantage. That is, the adjustment holes in the band are only provided at predetermined intervals. Therefore, when the length of the band is adjusted, the fastening strength varies, and this degrades the measurement accuracy.

The technology disclosed in JP-2003-220041A poses a disadvantage of complicated band structure. The technology poses another disadvantage. That is, when the band and the sensor main unit are not kept in balance, the pulse wave sensor becomes prone to be displaced.

### SUMMARY OF THE INVENTION

The present invention addresses the above disadvantages. Therefore, it is an objective of the present invention to provide a body condition measuring device that is of a simple structure and can be favorably attached to a living body.

To achieve the objective of the present invention, there is provided a body condition measuring device, which includes a sensor main unit and a band. The sensor main unit has a measurement arrangement. The measurement arrangement contacts a living body and measures a condition of the living body. The band is wrapped around an attachment region of the living body and attaches the sensor main unit to the attachment region of the living body. The sensor main unit has at least one band anchoring portion. Each of the at least one band anchoring portion is positioned at a corresponding one of opposed ends of the sensor main unit and has a band insertion hole for receiving the band therethrough. The band is substantially made of a flexibly deformable elastic material and is anchored in the band insertion hole of each of the at least one band anchoring portion due to an elastic force generated upon deformation of the band. The band urges the measurement arrangement against the living body due to the elastic force generated upon deformation of the band.

### BRIEF DESCRIPTION OF THE DRAWINGS

The invention, together with additional objectives, features and advantages thereof, will be best understood from the following description, the appended claims and the accompanying drawings in which:

FIG. 1 is an explanatory view illustrating a pulse wave sensor in a first embodiment of the present invention;

FIG. 2 is an explanatory view illustrating the way a band is passed through band anchoring portions in the first embodiment;

FIG. 3 is an explanatory view illustrating change in the band between before and after attachment in the first embodiment;

FIG. 4 is an explanatory view illustrating the pulse wave sensor in the first embodiment as is attached;

FIG. 5A is an explanatory view illustrating an installed state of the pulse wave sensor of the first embodiment around a hand;

FIG. 5B is an explanatory view illustrating a circled portion VB in FIG. 5A and a system configuration of the pulse wave sensor in the first embodiment;

FIG. 6A is an explanatory view illustrating a pulse wave sensor in a second embodiment;

FIG. 6B is an explanatory view illustrating part of the sensor as is enlarged;

FIG. 7 is an explanatory view illustrating a pulse wave sensor in a third embodiment;

FIG. 8 is an explanatory view illustrating the usage of the pulse wave sensor in the third embodiment;

FIG. 9A is an explanatory view illustrating a pulse wave sensor in a fourth embodiment;

FIG. 9B is an enlarged view of a circled portion IXB in FIG. 9A seen from a different direction;

FIG. 9C is an explanatory view illustrating a modification to the fourth embodiment;

FIG. 10A is an explanatory view illustrating a pulse wave sensor in a fifth embodiment;

FIG. 10B is an explanatory view illustrating a modification to the fifth embodiment;

FIG. 11 is an explanatory view illustrating a pulse wave sensor in a sixth embodiment;

FIG. 12 is an explanatory view illustrating a pulse wave sensor in a seventh embodiment;

FIG. 13 is an explanatory view illustrating a pulse wave sensor in an eighth embodiment;

FIG. 14A is an explanatory view illustrating a pulse wave sensor in a ninth embodiment;

FIG. 14B is an explanatory view illustrating a pulse wave sensor in tenth embodiment;

FIG. 14C is an explanatory view illustrating a pulse wave sensor in an eleventh embodiment;

FIG. 15A is an explanatory view illustrating a pulse wave sensor in a twelfth embodiment;

FIG. 15B is an explanatory view illustrating a modification to the twelfth embodiment;

FIG. 15C is an explanatory view illustrating another modification to the twelfth embodiment;

FIG. 16A is an explanatory view illustrating a pulse wave sensor in a thirteenth embodiment;

FIG. 16B is an explanatory view illustrating the main unit of the pulse wave sensor;

FIG. 17A is an explanatory view illustrating a pulse wave sensor in a fourteenth embodiment;

FIG. 17B is an explanatory view illustrating a modification to the fourteenth embodiment;

FIG. 18A is an explanatory view illustrating a pulse wave sensor in a fifteenth embodiment;

FIG. 18B is an explanatory view illustrating the band of the pulse wave sensor;

FIG. 19A is an explanatory view illustrating a pulse wave sensor in a sixteenth embodiment;

FIG. 19B is an explanatory view illustrating a modification to the sixteenth embodiment;

FIG. 20 is an explanatory view showing another modification to the sixteenth embodiment as viewed from the inner band portion side;

FIG. 21A is an explanatory view illustrating a pulse wave sensor in a seventeenth embodiment; and

FIG. 21B is an explanatory view illustrating the usage of the pulse wave sensor.

### DETAILED DESCRIPTION OF THE INVENTION

Description will be given to the best modes for carrying out the present invention (embodiments).

### First Embodiment

In the following description, a pulse wave sensor that is attached around a wrist of a human body and detects pulse waves will be taken as an example of a body condition measuring device.

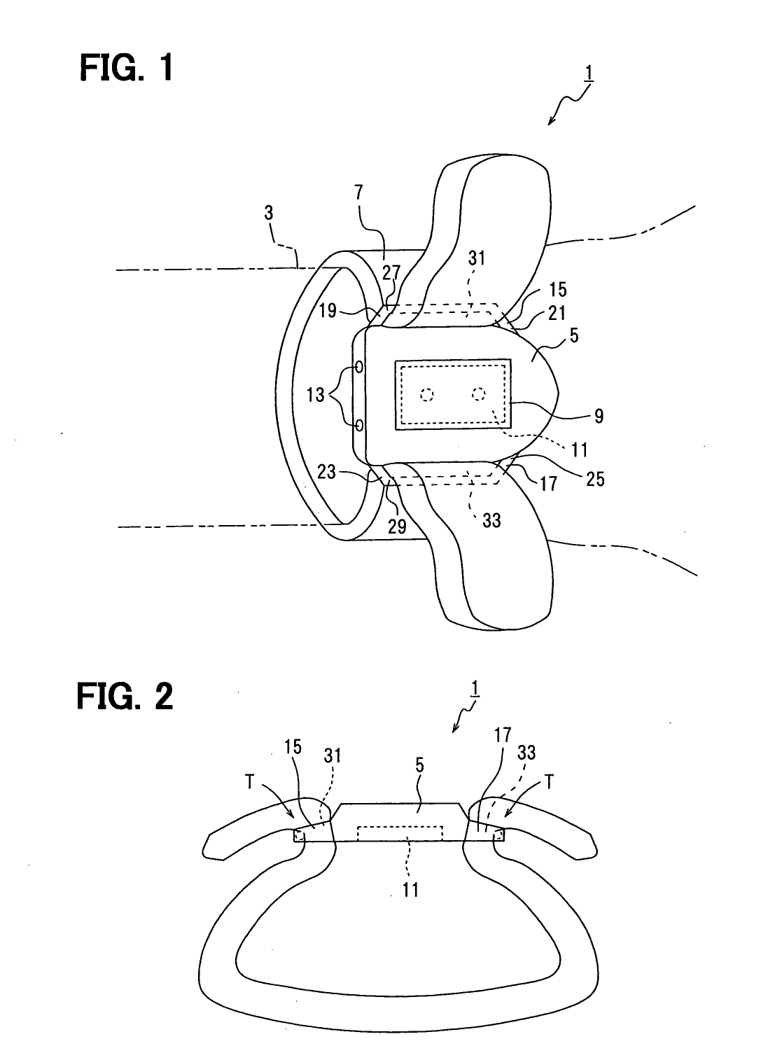

(a) First, description will be given to the configuration of the pulse wave sensor in this embodiment with reference to FIG. 1.

As illustrated in FIG. 1, the pulse wave sensor 1 in this embodiment is a wrist watch-type sensor so designed that it is attached around, for example, a wrist 3 of a human body. The pulse wave sensor 1 includes a pulse wave sensor main unit 5 and a band 7. The band 7 attaches the pulse wave sensor main unit 5 around the wrist 3.

The pulse wave sensor main unit 5 is a plate-like member, which is shaped into generally a boat form having a tapered end. The pulse wave sensor main body 5 protrudes in the direction of the end of the hand (to the right in FIG. 1). That is, the sensor main unit 5 is asymmetric in the left-right direction of FIG. 1 (the extending direction of the hand, i.e., the direction of the axis of the pulse wave sensor main unit 5). At the same time, the sensor main unit 5 is symmetric in the top-bottom direction of FIG. 1 (the circumferential direction of the wrist 3).

The pulse wave sensor main unit 5 is provided on its surface with a liquid crystal display portion (monitor) 9, and on its rear face with a measurement arrangement 11 for detecting pulse waves. Operating switches 13 for operating, for example, turning on and off the pulse wave sensor 1 are provided at a side end of the pulse wave sensor main unit 5 in the axial direction (left side in FIG. 1).

Two band anchoring portions 15 and 17, through which the band 7 is passed, are provided on the pulse wave sensor main unit 5 to oppose one another in the vertical direction of FIG. 1 (the circumferential direction of the wrist 3). The band anchoring portions 15 and 17 are provided so that they are protruded outward. The band anchoring portions 15 and 17 are members that comprise left and right legs 19 to 25 and beams 27 and 29 that connect both the legs 19 to 25, and are substantially trapezoidal in outer shape. Strip-shaped band insertion holes 31 and 33 through which the band 7 is passed are formed so that they are encircled with both the legs 19 to 25, beams 27 and 29, and pulse wave sensor main unit 5.

The band 7 is an elongated strip-shaped body having elasticity. The band 7 is made of a rubber material, a casual rubber material and/or a pile fabric material. The rubber material is formed of rubber material woven of rubber strings. The casual rubber is covered with a cloth material. The pile fabric material (like supporters) is covered with towel cloth, or the like. In sum, the band 7 is formed of a material such as rubber that is flexibly deformed when external force is applied, and is restored to its original shape when external force is removed.

The length of the band 7 is 150 mm, for example. Thus, when the band 7 is wrapped around the wrist 3 and passed through the band insertion holes 31 and 33, a sufficient length (e.g. 100 mm) is left. The thickness of the band 7 is so set that it is larger by, for example, 2 mm or so than the dimension of the band insertion holes 31 and 38 in the direction of their shorter sides (the vertical direction in FIG. 1). The width of the band 7 is so set that it is slightly larger, for example, by 10 mm or so than the dimension (e.g. 20 mm) of the band insertion holes 31 and 33 in the direction of their longer sides (the horizontal direction in FIG. 1).

(b) Description will be given to how to attach the pulse wave sensor 1 constructed as mentioned above around the wrist 3.

First, as illustrated in FIG. 2, both the longitudinal ends of the band 7 are passed through the band insertion holes 31 and 33 in the band anchoring portions 15 and 17 of the pulse wave sensor main unit 5. At this time, both the ends of the band 7 are passed from the inner side (the rear side on which the measurement arrangement 11 is positioned) of the pulse wave sensor main unit 5 to the outer side. Thus, the band 7 is brought into ring shape.

The user passes his/her hand through the ring of the band 7 in this state, and positions the pulse wave sensor 1 at his/her wrist 3.

The user pulls the ends (free ends) of the band 7, and shortens and tightens the ring of the band 7 while adjusting the degree to which the band 7 is tightened (state of pressing and state of contact). Thus, the pulse wave sensor 1 is attached around the wrist 3.

The outer dimension (the thickness and/or width) of the band 7 is larger than the corresponding inner dimension of the band insertion holes 31 and 33. Therefore, the band 7 is compressed in the band insertion holes 31 and 33. After the band 7 is passed through the band insertion holes 31 and 33, the outer shape of the band is substantially restored to its original state by the elasticity of the band 7 itself. Thus, the band 7 is in its substantially original dimensions in proximity to (in positions in front of and behind) the band insertion holes 31 and 33. This prevents the attachment positions T of the band 7 from being shifted and the band 7 itself from coming off the band insertion holes 31 and 33.

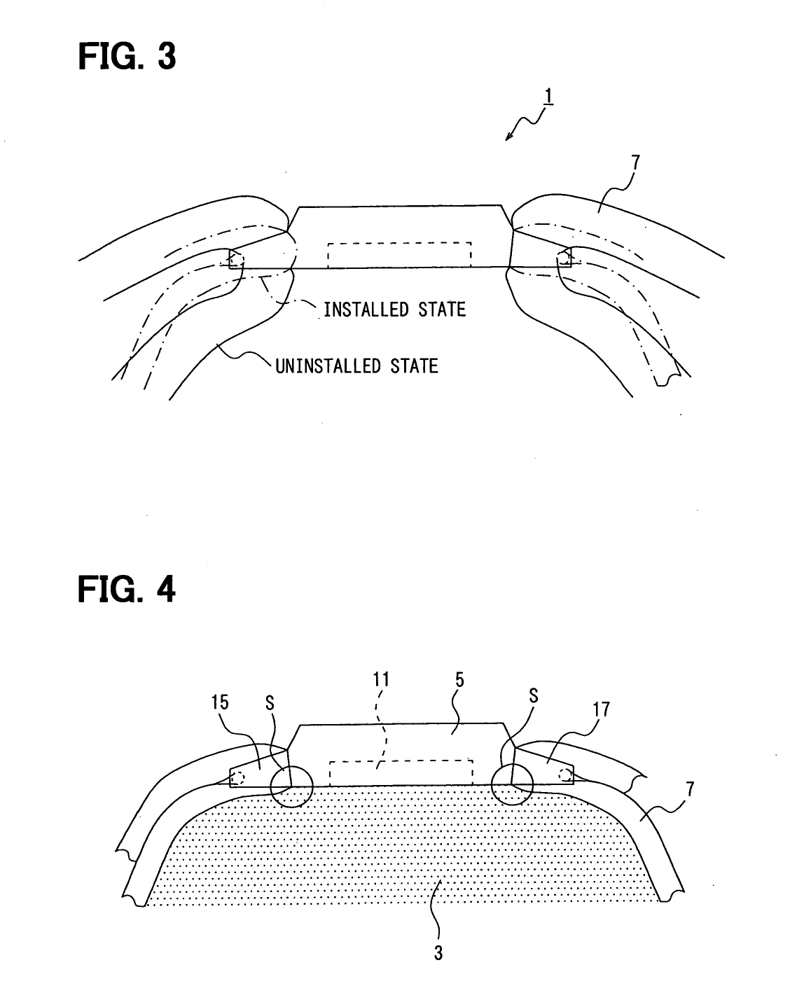

After the degree to which the band 7 is tightened is adjusted and the band 7 is attached to the pulse wave sensor main unit 5, the pulse wave sensor 1 may be removed from or thereafter reattached around the wrist 3. In this case, the pulse wave sensor is removed or reattached by pulling the stretchy band 7 to widen the ring without shifting the attachment positions T of the band 7. At this time, as illustrated in FIG. 3, the shape of the band 7 is changed between the installed state of the pulse wave sensor 1 to the wrist 3 and the uninstalled state of the pulse wave sensor 1 to the wrist 3. Specifically, the inner diameter of the ring of the band 7 is reduced when the pulse wave sensor 1 is uninstalled from the wrist 3 in comparison to the installed state of the pulse wave sensor 1.

Thus, the pulse wave sensor 1 (and thus the pulse wave sensor main unit 5) can be firmly attached around the wrist 3 by the elastic force of the band 7.

More specific description will be given. As illustrated in FIG. 4, the joints S in proximity to the bases of the band anchoring portions 15 and 17 is brought into tight contact with the surface of the wrist 3 without gaps therebetween. More specifically, the joints S are those between the inner surface of the band 7 and the inner surface of the pulse wave sensor main unit. As a result, the pulse wave sensor main unit 5 (especially, the measurement arrangement 11) can be fixed without displacement.

(c) Description will be given to the internal structure, operation, and the like of the pulse wave sensor 1 in this embodiment.

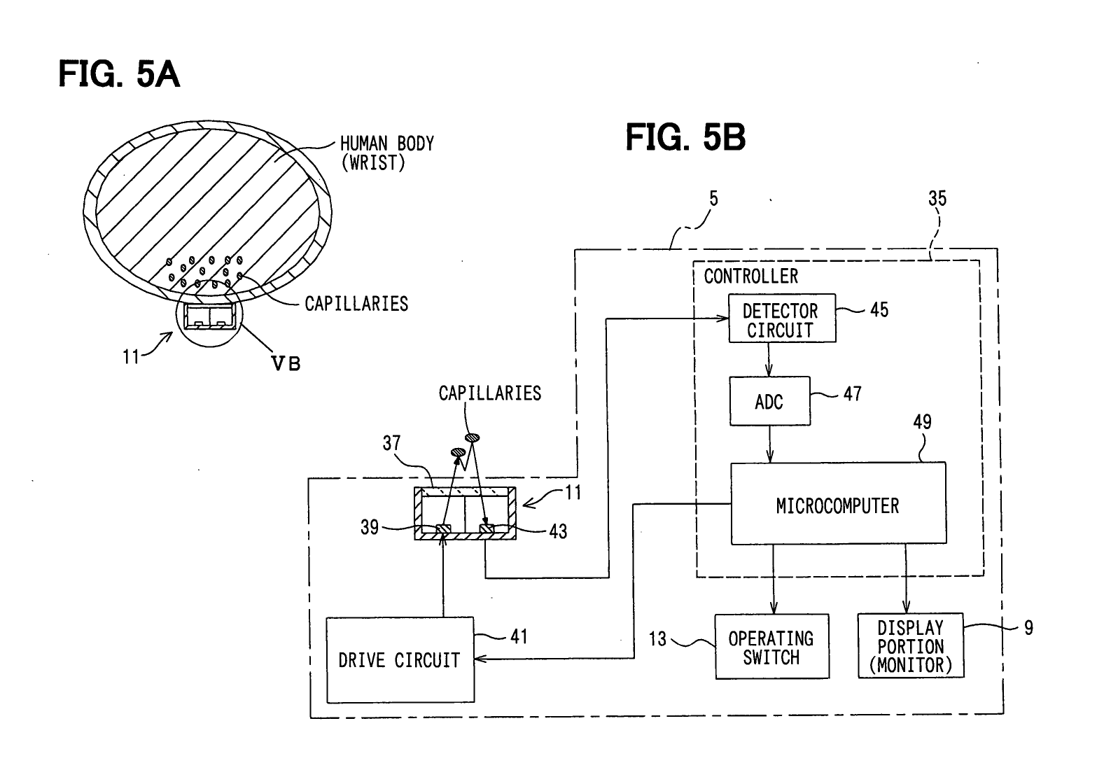

As illustrated in FIGS. 5A and 5B, the pulse wave sensor main unit 5 includes the measurement arrangement 11 and a control arrangement 35 that controls the measurement arrangement 11 and performs like operations. The pulse wave sensor main unit 5 is attached to the wrist so that the transparent window 37 in the measurement arrangement 11 is brought into tight contact with the wrist.

The measurement arrangement 11 is a publicly known optical reflective sensor having a light emitting device (e.g. light emitting diode: LED) 39, a drive circuit 41, a photoreceptor device (e.g. photodiode: PD) 43, and the transparent window 37 for passing the light.

This measurement arrangement 11 operates as follows. Light is projected from the light emitting device 39 to a human body. Part of the light impinges upon capillary arteries (capillaries) running in the human body, and is absorbed into hemoglobin in the blood flowing in the capillary arteries. The rest of the light is reflected and scattered by the capillary arteries, and part of it enters the photoreceptor device 43. At this time, the amount of hemoglobin in the capillary arteries is varied in a pulse-like fashion by pulsation of the blood. Therefore, the amount of light absorbed into hemoglobin is also varied in a pulse-like fashion. As a result, the amount of light reflected by the capillary arteries and detected at the photoreceptor device 43 varies. This variation in the amount of received light is outputted as pulse wave information (e.g. voltage signal) to the control arrangement 35.

The control arrangement 35 is provided with functions as a pulse wave analyzer, and has therein a detector circuit 45, ADC (AD Converter) 47, and a microcomputer 49.

The control arrangement 35 is connected with an input section and a display portion. The input section (i.e. operating switches) 13 is for inputting varied data (manually or by any other means), and the display portion (i.e. monitor) 9 is for displaying the result of detection and the like.

(d) Description will be given to the effects of this embodiment.

In this embodiment, the pulse wave sensor 1 is so constructed that it is fixed by passing an elastic long strip-shaped band 7 of simple structure through band anchoring portions 15 and 17 at both its ends. Owing to the elasticity of the band 7, the pulse wave sensor 1 can be fixed without displacement no matter where the pulse wave sensor 1 is attached on the wrist 3 or the like.

In this embodiment, the outer dimensions of the band 7 are larger than the inner dimensions of the band insertion holes 31 and 33. Therefore, the band 7 can be anchored at the band anchoring portions 15 and 17 in predetermined attachment positions T only by passing the band 7 through the band insertion holes 31 and 33. This is done by the elastic force of the band 7 itself. Thus, the band 7 is not displaced from the attachment positions T or does not come off regardless of whether the pulse wave sensor 1 is attached or removed.

The attachment positions T of the band 7 can be changed by pulling both the ends of the band. (That is, the lengths of both the ends are adjustable.) This brings the advantage that pressing force and the like can be adjusted with ease.

In this embodiment, the joints S between the bases of the band anchoring portions 15 and 17 and the band 7 are in tight contact with the surface of the wrist 3 without gaps in-between. Therefore, the pulse wave sensor 1 can be firmly fixed without displacement (without wobbling). As a result, the measurement accuracy can be enhanced.

The band 7 is so constructed that it is passed through the band anchoring portions 15 and 17 from inside to outside. In this respect as well, the gaps can be eliminated between the pulse wave sensor main unit 5 and the wrist 3.

In this embodiment, the band is not fixed using a plurality of fixing holes like conventional wristwatch bands. The length of the band 7 in this embodiment is linearly adjustable, and this brings the following advantages. That is, fastening strength is less prone to vary, and the measurement accuracy is less prone to be degraded.

In this embodiment, once the band 7 has been attached, the pulse wave sensor 1 is attached or removed by widening the ring of the band 7. Therefore, the attachment positions T of the band 7 are not shifted. As a result, the measurement arrangement 11 can be consistently pressed with constant pressing force, and the constant measurement accuracy can be maintained.

In this embodiment, the pulse wave sensor main unit 5 is horizontally asymmetric, and this brings the advantage that the direction of attachment can be learnt at a glance. Apart from this, the pulse wave sensor main unit 5 may be formed in vertically asymmetric shape. When the direction of attachment is reverse, the positions of the light emitting device 39 and the photoreceptor device 43 of the measurement arrangement 11 are shifted. Therefore, the same measurement conditions are not obtained, and this can lead to deviation in the result of measurement. This is not preferable.

### Second Embodiment

Description will be given to a pulse wave sensor in a second embodiment. The same description as of the first embodiment will be omitted.

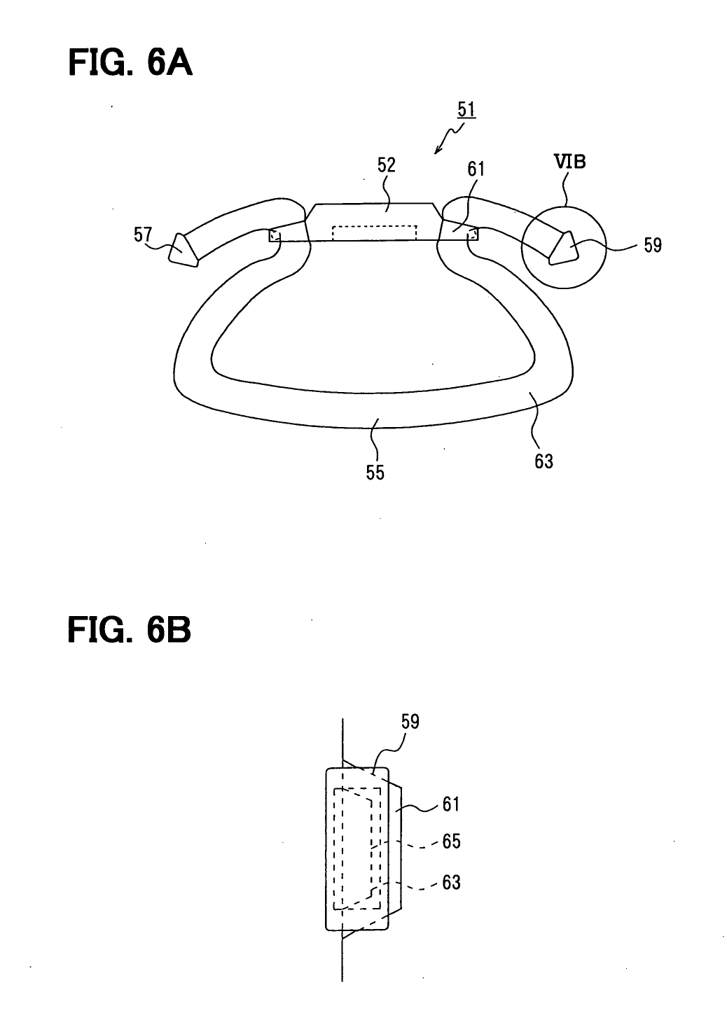

As illustrated in FIG. 6A, the pulse wave sensor 51 in this embodiment comprises a plate-like pulse wave sensor main unit 52 and an elastic long strip-shaped band 55, like that in the first embodiment.

In this embodiment, especially, the band 55 is provided at its both ends with substantially triangular prismatic projections 57 and 59.

As illustrated in FIG. 6B, these projections 57 and 59 are larger in outer dimensions than the main part 63 of the long strip-shaped band. (FIG. 6B is a schematic diagram wherein the portion marked with “VIB” in FIG. 6A is disposed over the band anchoring portion 61 for the purpose of comparison of size.) Thus, the dimensions of the projections 57 and 59 are considerably larger than the inner dimensions of the band insertion holes 65 in the band anchoring portions 61, and the projections 57 and 59 jut out.

In this embodiment, therefore, the band 55 does not come off the band anchoring portions 61 even when the band 55 is pulled hard.

The constitution for preventing the band 55 from coming off, like the projections 57 and 59, may be provided only at one end, not at both ends. The projections 57 and 59 may be formed by machining the ends of the band 55, or may be formed by joining or connecting other members. The shape of the projections 57 and 59 is not limited to substantially triangular prismatic shape.

### Third Embodiment

Description will be given to a pulse wave sensor in a third embodiment. The same description as of the first embodiment will be omitted.

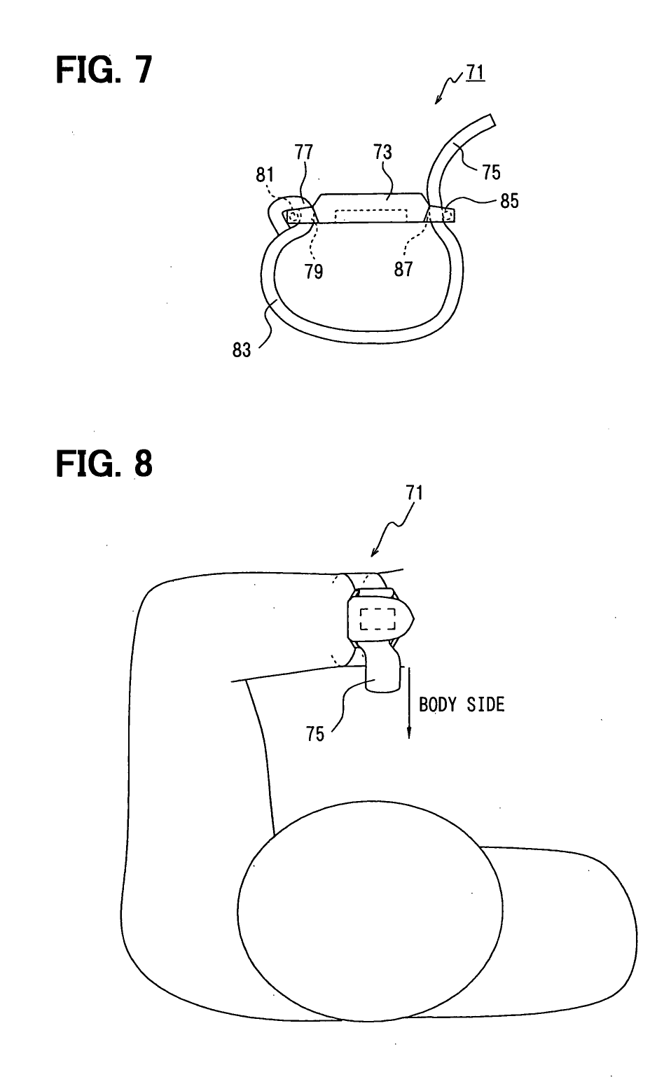

As illustrated in FIG. 7, the pulse wave sensor 71 in this embodiment comprises a plate-like pulse wave sensor main unit 73 and an elastic long strip-shaped band 75, like that in the first embodiment.

In this embodiment, especially, one end (the left end in the figure) of the band 75 is fixed in proximity to one band anchoring portion 77. More specific description will be given. One end (fixed end) of the band 75 is passed through the band insertion hole 79 and looped around a pin 81. In this state, the one end is secured on the outer surface of the band main part 83 by sewing, bonding, or the like.

The other end (the right end in the figure) of the band 75 is passed through the band insertion hole 87 in the other band anchoring portion 85. The degree to which the band 75 is tightened can be adjusted by pulling the other end (free end).

In this embodiment, therefore, the degree to which the band 75 is tightened can be adjusted only by pulling the free end of the band 75. Since the band 75 is fixed at the fixed end, the band 75 does not come off the one band anchoring portion 77 even when the free end is pulled hard.

In this embodiment, especially, the free end of the band is positioned in front (on the body side) of a person who wears the pulse wave sensor 71, as illustrated in FIG. 8. More specifically, the free end of the band is located on a thumb side of the wrist of the person, which is opposite from a little finger side of the wrist. This brings the advantage that the degree to which band 75 is tightened can be adjusted with ease.

### Fourth Embodiment

Description will be given to a pulse wave sensor in a fourth embodiment. The same description as of the first embodiment will be omitted.

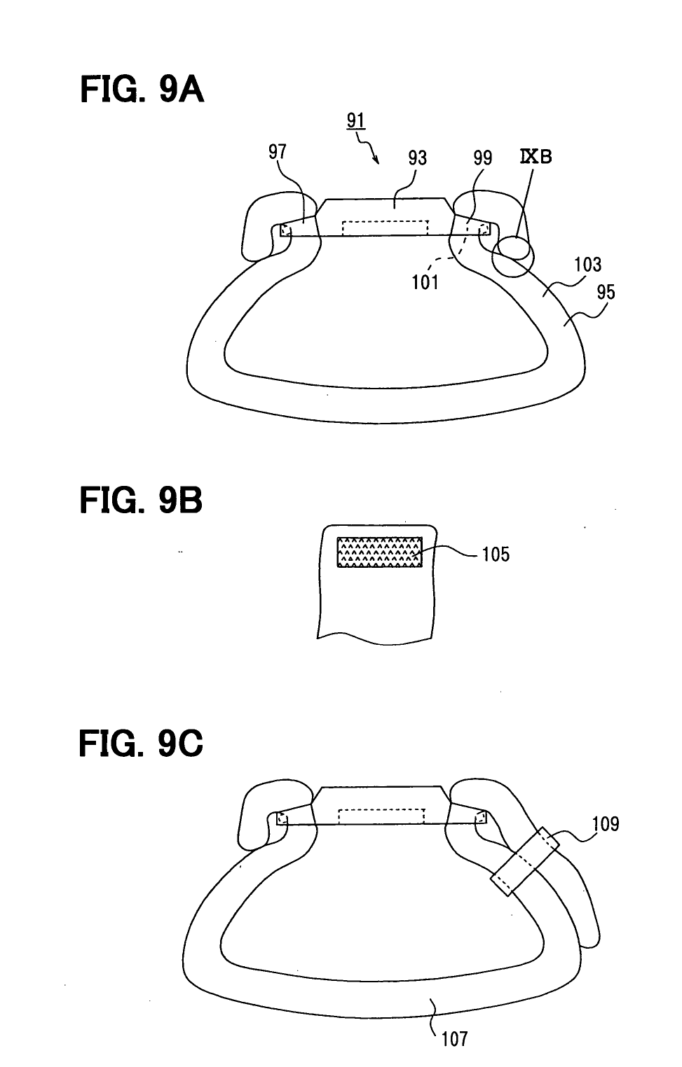

As illustrated in FIGS. 9A and 9B, the pulse wave sensor 91 in this embodiment includes a plate-like pulse wave sensor main unit 93 and an elastic long strip-shaped band 95, like that in the first embodiment.

In this embodiment, especially, one end (the left end in the figure) of the band 95 is fixed as in the third embodiment. Specifically, the one end of the band 95 is secured on the surface of the band 95 in proximity to one band anchoring portion 97 by sewing, bonding, or the like.

The other end (the right end in the figure) of the band 95 is passed through the band insertion hole 101 in the other band anchoring portion 99. The other end (free end) is detachably attached to the outer surface of the band main part 103.

More specific description will be given. For example, as shown in FIG. 9B, a hook-and-loop fastener 105 is provided on the outer surface of the band main part 103 and at the free end of the band 95. The outer surface of the band main part 103 and the free end of the band 95 are detachably joined with each other by this hook-and-loop fastener 105.

In this embodiment, therefore, the free end of the band 95 is prevented from largely protruding from the pulse wave sensor main unit 93 into the surrounding space. The free end is integrated with the pulse wave sensor 1 in a compact manner. This brings the advantage of the enhanced ease of use of the pulse wave sensor 1.

As illustrated in FIG. 9C, a ring member 109 may be fit onto the band 107, and the free end of the band 107 may be fastened by this ring member 109.

### Fifth Embodiment

Description will be given to a pulse wave sensor in a fifth embodiment. The same description as of the first embodiment will be omitted.

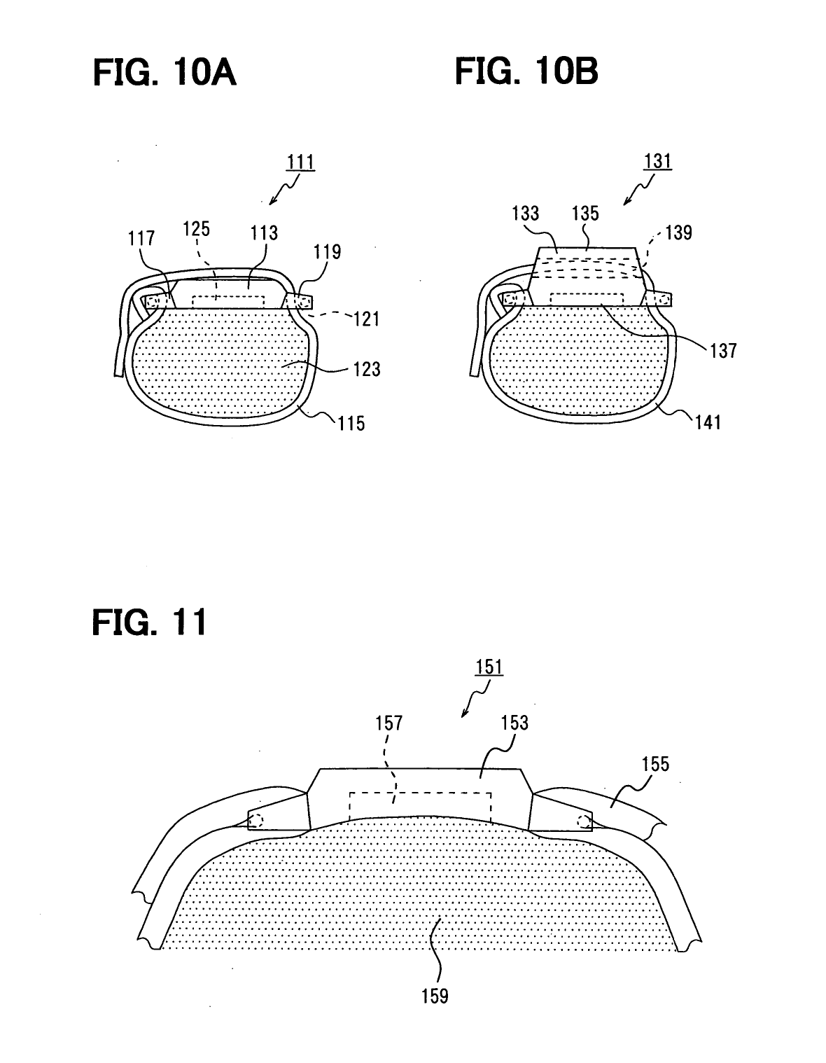

As illustrated in FIG. 10A, the pulse wave sensor 111 in this embodiment comprises a plate-like pulse wave sensor main unit 113 and an elastic long strip-shaped band 115, like that in the first embodiment.

In this embodiment, one end of the band 115 is secured on the surface of the band 115 in proximity to one band anchoring portion 117 as in the fourth embodiment.

The other end (free longitudinal end) of the band 115 is passed through the band insertion hole 121 in the other band anchoring portion 119, and is disposed so that the surface (an outer side) of the pulse wave sensor main unit 113 is covered with the band 115. The free end of the band 115 is detachably fastened to the outer surface of the band 115 on the one band anchoring portion 117 side of the pulse wave sensor main unit 113 by a hook-and-loop fastener or the like.

Thus, the pulse wave sensor main unit 115 is pressed toward the wrist 123 from its surface side, and is firmly fixed. As a result, the measurement accuracy can be enhanced.

In this embodiment, there are possible cases where the pulse wave sensor main unit 115 is not provided on its surface with a monitor. In this case, the following structure can be adopted. That is, data may be stored in the memory of the measurement arrangement 125 and may be thereafter outputted as required. Needless to add, a monitor may be provided. In this case, the free end of the band 115 is unfastened when the monitor is viewed.

Apart from this, the constitution illustrated in FIG. 10B may be adopted for the pulse wave sensor 131.

More specific description will be given. The pulse wave sensor main unit 133 is provided with a monitor 135 and a measurement arrangement 137. Further, a through hole 139 is provided between the monitor 135 and the measurement arrangement 137 in parallel with the surface of the pulse wave sensor main unit 133 (in the horizontal direction in the figure). The band 141 is passed through this through hole 139.

This brings the following advantages. That is, the pulse wave sensor main unit 133 can be firmly fixed, and the monitor 135 is constantly viewable.

### Sixth Embodiment

Description will be given to a pulse wave sensor in a sixth embodiment. The same description as of the first embodiment will be omitted.

As illustrated in FIG. 11, the pulse wave sensor 151 in this embodiment comprises a plate-like pulse wave sensor main unit 153 and an elastic long strip-shaped band 155, like that in the first embodiment.

In this embodiment, especially, the rear side of the pulse wave sensor main unit 153 is curved in correspondence with the curve in the wrist 159 in the circumferential direction (the horizontal direction in the figure). The rear side of the pulse wave sensor main unit 153 is the side on which the measurement arrangement 157 is positioned and is brought into contact with the wrist 159 (the lower side in the figure). No curve is specially provided in the direction of the length of the wrist 159 (the direction of the depth of the figure).

Thus, the pulse wave sensor main unit 153 (and thus the measurement arrangement 157) is brought into tight contact with the wrist 159 without gaps in-between. This brings about the following effects. That is, the pulse wave sensor main unit 153 is less prone to be displaced, and the measurement accuracy is enhanced.

### Seventh Embodiment

Description will be given to a pulse wave sensor in a seventh embodiment. The same description as of the first embodiment will be omitted.

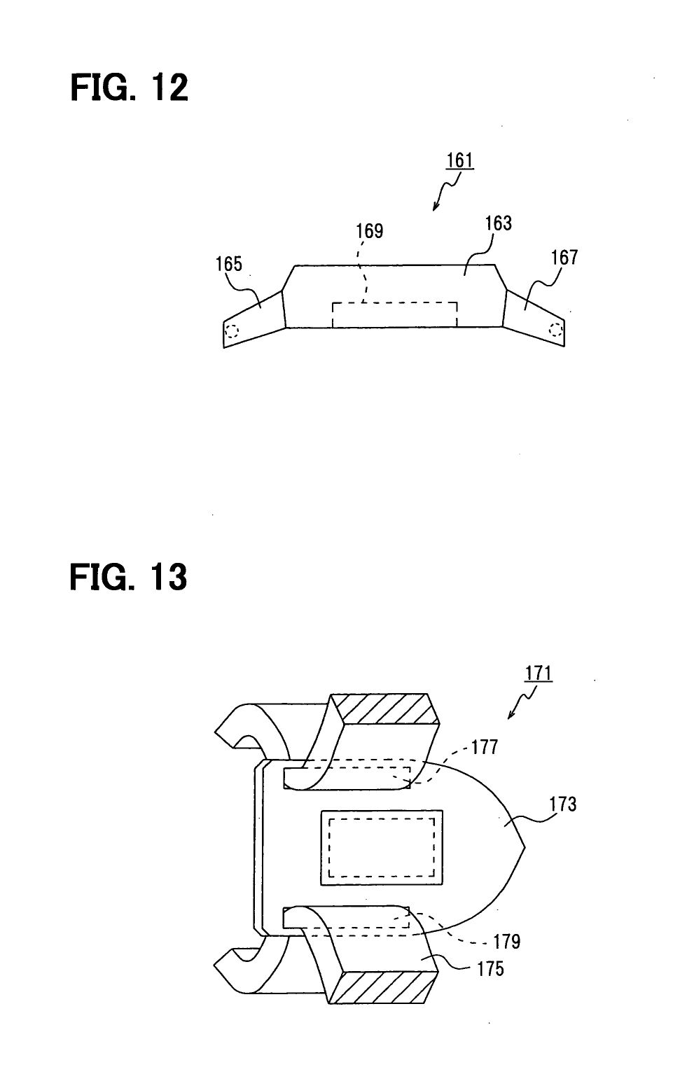

As illustrated in FIG. 12, the pulse wave sensor 161 in this embodiment is so constructed that the following is implemented. That is, band anchoring portions 165 and 167 are provided at both the ends (the left and right ends in the figure) of the pulse wave sensor main unit 163, and the band anchoring portions 165 and 167 are protruded to the areas between the rear surface (the surface positioned on the lower side in the figure) of the pulse wave sensor main unit 163 and the wrist (to be positioned at the lower part of the figure).

Thus, the shape of the pulse wave sensor main unit 163 and the band anchoring portions 165 and 167 is analogous to the circumferential shape of the wrist, as in the sixth embodiment. Therefore, the pulse wave sensor main unit 163 (and thus the measurement arrangement 169) is brought into tight contact with the wrist. This brings about the following effects. That is, the pulse wave sensor main unit 163 is less prone to be displaced, and the measurement accuracy is enhanced.

### Eighth Embodiment

Description will be given to a pulse wave sensor in an eighth embodiment. The same description as of the first embodiment will be omitted.

As illustrated in FIG. 13, the pulse wave sensor 171 in this embodiment comprises a plate-like pulse wave sensor main unit 173 and an elastic long strip-shaped band 175, like that in the first embodiment.

In this embodiment, especially, band insertion sections 177 and 179 are positioned inward of the outer perimeter of the pulse wave sensor main unit 173.

This brings the following advantage unlike cases where band anchoring portions that are protruded outward from the pulse wave sensor main unit 173 are provided. That is, situations, in which something hits either of the band anchoring portions, and the pulse wave sensor main unit 173 is displaced, do not occur. Thus, this embodiment brings about the following effects. That is, the pulse wave sensor main unit 173 is less prone to be displaced, and the measurement accuracy is enhanced.

### Ninth Embodiment

Description will be given to a pulse wave sensor in a ninth embodiment. The same description as of the first embodiment will be omitted.

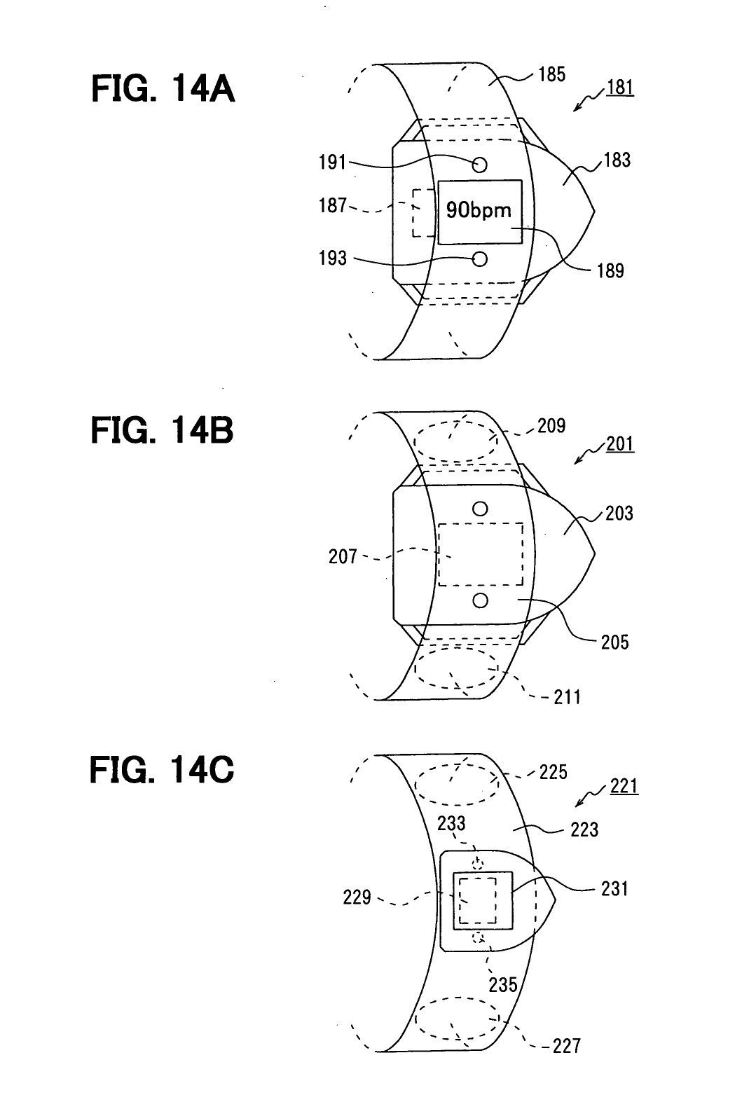

As illustrated in FIG. 14A, the pulse wave sensor 181 in this embodiment comprises a plate-like pulse wave sensor main unit 183 and an elastic long strip-shaped band 185, like that in the first embodiment.

In this embodiment, especially, the pulse wave sensor main unit 183 is provided with a measurement arrangement 187, and the band 185 is provided with a monitor 189. The measurement arrangement 187 and the monitor 189 are mechanically and electrically connected with each other by, for example, snap-like connecting portions 191 and 193.

In this case, the band 185 may be formed in ring shape, or may be made annular by fastening it on the side opposite the pulse wave sensor main unit 183 (on the opposite side with respect to the wrist) by a hook-and-loop fastener or the like.

This embodiment is so constructed that the pulse wave sensor main unit 183 is covered with the band 185 and is thereby pressed against the wrist. This brings the advantage that the pulse wave sensor main unit 183 is less prone to be displaced.

### Tenth Embodiment

Description will be given to a pulse wave sensor in a tenth embodiment. The same description as of the ninth embodiment will be omitted.

As illustrated in FIG. 14B, the pulse wave sensor 201 in this embodiment comprises a plate-like pulse wave sensor main unit 203 and an elastic long strip-shaped band 20, like that in the ninth embodiment.

In this embodiment, especially, the pulse wave sensor main unit 203 is provided with a measurement arrangement 207 but the band 205 is not provided with a monitor. In addition, batteries 209 and 211 are embedded in the band 205.

This aspect of the present invention is so constructed that the batteries 209 and 211 are embedded in the band 205, and the pulse wave sensor main unit 203 is covered with the band 205 and is thereby pressed against the wrist. This brings the advantage that the pulse wave sensor main unit 203 is further less prone to be displaced.

### Eleventh Embodiment

Description will be given to a pulse wave sensor in an eleventh embodiment. The same description as of the tenth embodiment will be omitted.

As illustrated in FIG. 14C, the pulse wave sensor 221 in this embodiment is so constructed that batteries 225 and 227 are embedded in an elastic long strip-shaped band 223, like that in the tenth embodiment. In addition, the band 223 is provided with a measurement arrangement 229.

As illustrated in the figure, a separate monitor 231 may be mounted through connecting portions 233 and 235. Instead, a monitor itself may be provided in the band 223. Alternatively, the monitor 231 may be omitted. Operating switches and the like may be disposed in the band 223.

### Twelfth Embodiment

Description will be given to a pulse wave sensor in a twelfth embodiment. The same description as of the first embodiment will be omitted.

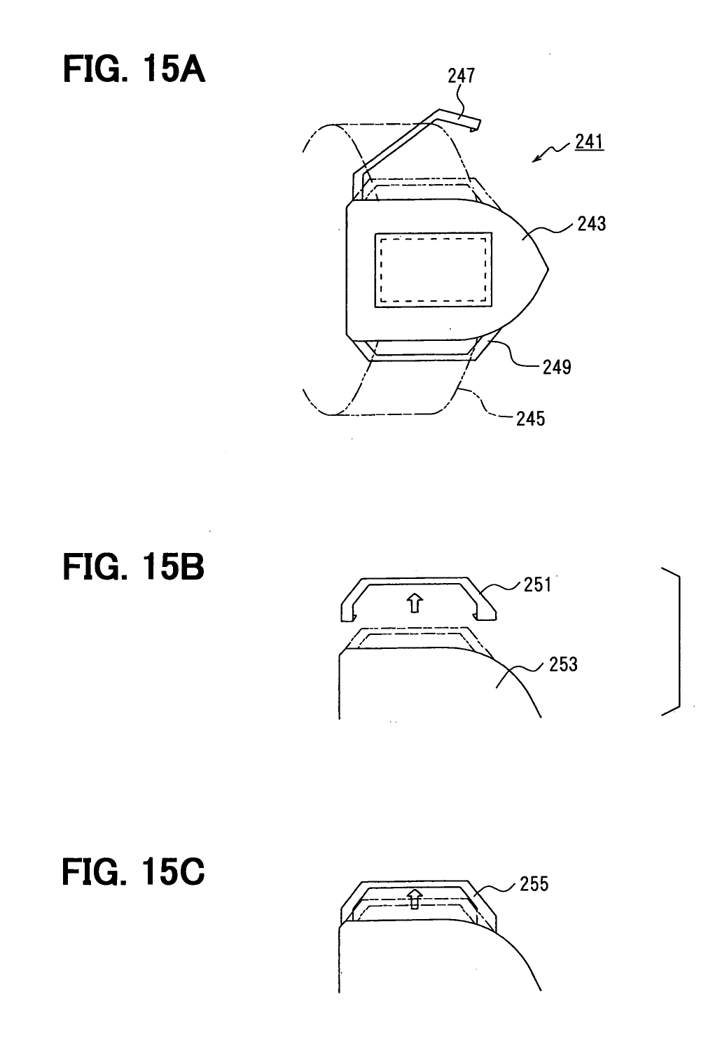

As illustrated in FIG. 15B, the pulse wave sensor 241 in this embodiment comprises a plate-like pulse wave sensor main unit 243 and an elastic long strip-shaped band 245, like that in the first embodiment.

In this embodiment, especially, either or both of the band anchoring portions 247 and 249 installed on the pulse wave sensor main unit 245 can be caused to pivot.

Therefore, when the band 245 is attached to, for example, the band anchoring portion 247, the band anchoring portion 247 is caused to pivot and opened, and is thereafter closed. This facilitates attachment.

Another example of this embodiment is illustrated in FIG. 15B. As in this example, the pulse wave sensor may be so constructed that the band anchoring portion 251 can be completely detached from the pulse wave sensor main unit 253.

A further example is illustrated in FIG. 15C. As in this example, the pulse wave sensor may so constructed that the band anchoring portion 255 can be slid in the direction of thickness of the band (vertical direction in the figure).

In any case mentioned above, when the band insertion hole is closed to fix the band anchoring portion 247, 251, or 255, it is fixed with the band compressed and deformed.

### Thirteenth Embodiment

Description will be given to a pulse wave sensor in a thirteenth embodiment. The same description as of the first embodiment will be omitted.

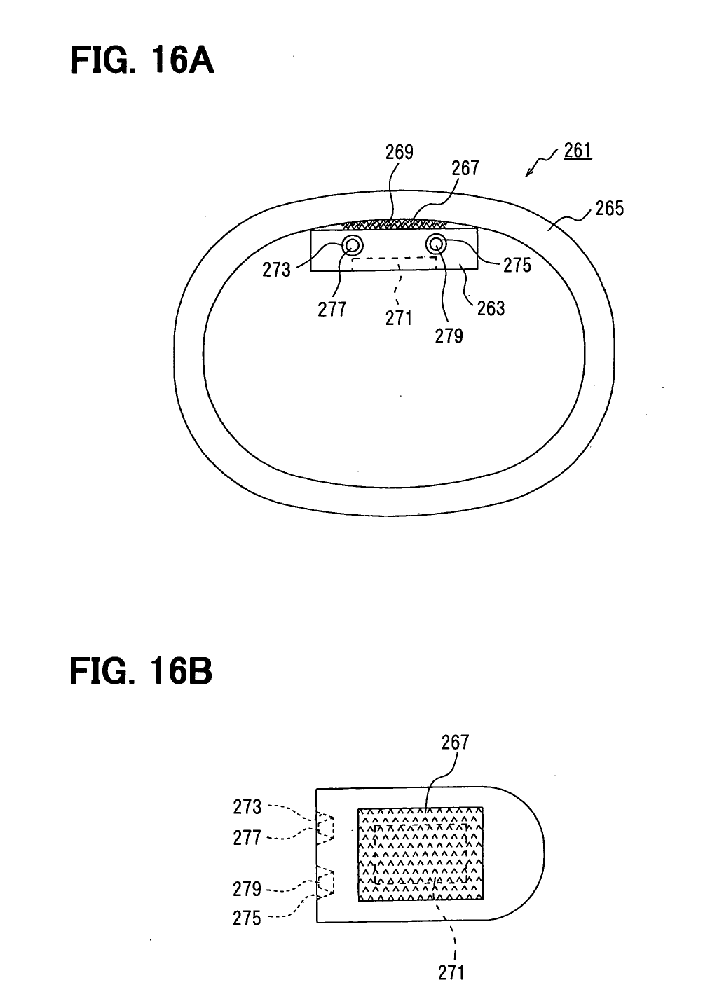

As illustrated in FIG. 16A, the pulse wave sensor 261 in this embodiment comprises a plate-like pulse wave sensor main unit 263 and an elastic long strip-shaped band 265, like that in the first embodiment.

In this embodiment, especially, the band 265 is annular, and a pulse wave sensor main unit 263 is secured to the inner side of the band 265 with hook-and-loop fasteners 267 and 269.

More specific description will be given. The hook-and-loop fastener 269 is provided on the inner surface of the band 265, and the hook-and-loop fastener 267 is provided on the upper face (measurement arrangement 271) of the pulse wave sensor main unit 263 as well. The pulse wave sensor main unit 263 is attached to the band 265 by both the hook-and-loop fasteners 267 and 269.

As illustrated in FIG. 16B, recesses 273 and 275 are formed at a side end of the pulse wave sensor main unit 263. Operating switches 277 and 279 are disposed in these recesses 273 and 275.

Thus, the band 265 and the pulse wave sensor main unit 263 can be integrally fixed with ease though they are separated from each other. Since the operating switches 277 and 279 are disposed in the recesses 273 and 275, the advantage that they are less prone to catch something is brought.

### Fourteenth Embodiment

Description will be given to a pulse wave sensor in a fourteenth embodiment. The same description as of the first embodiment will be omitted.

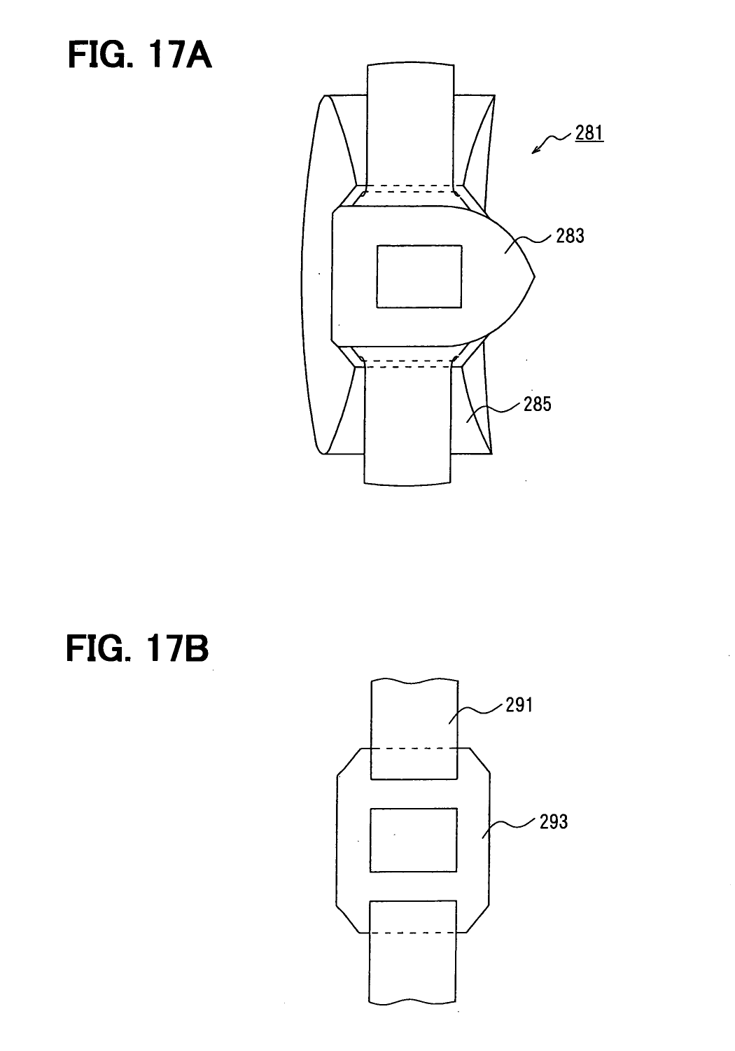

As described in FIG. 17A, the pulse wave sensor 281 in this embodiment comprises a plate-like pulse wave sensor main unit 283 and an elastic long strip-shaped band 285, like that in the first embodiment.

In this embodiment, especially, the band 285 is wider on the side opposite the pulse wave sensor main unit 283 (on the far side in the figure).

Thus, the pulse wave sensor 281 becomes less prone to be displaced, and the measurement accuracy can be enhanced.

The constitution illustrated in FIG. 17B may be adopted. In this constitution, the width of the band 291 itself is substantially constant, and a wider plate-like stabilizing member 293 is attached on the side opposite the pulse wave sensor main unit 283.

### Fifteenth Embodiment

Description will be given to a pulse wave sensor in a fifteenth embodiment. The same description as of the first embodiment will be omitted.

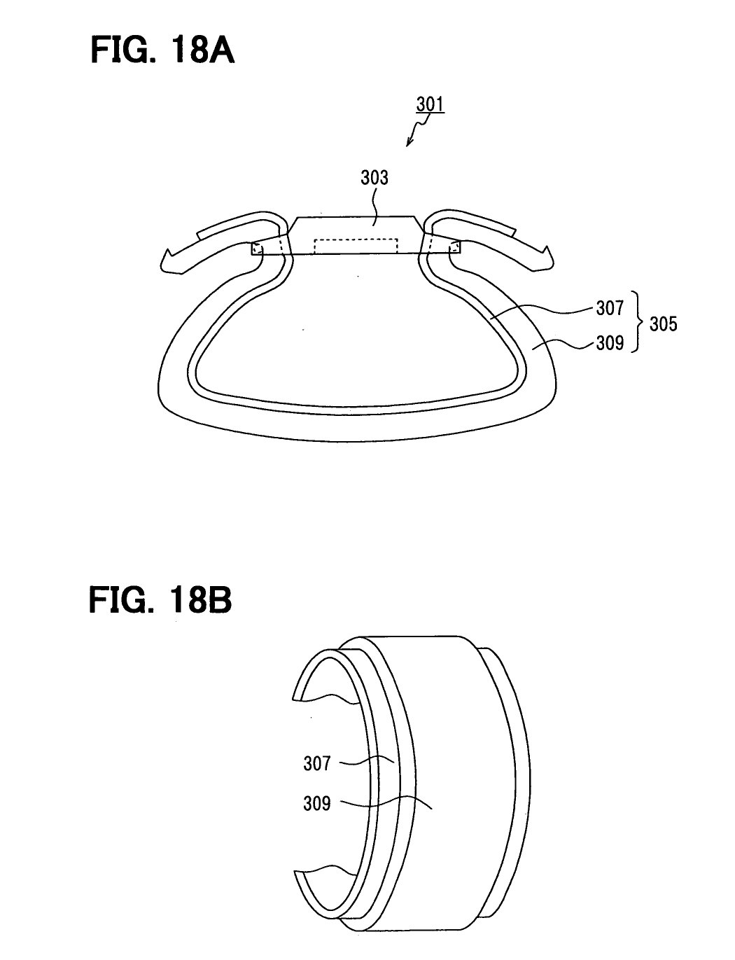

As illustrated in FIG. 18A, the pulse wave sensor 301 in this embodiment comprises a plate-like pulse wave sensor main unit 303 and an elastic long strip-shaped band 305, like that in the first embodiment.

In this embodiment, especially, the band 305 is of two-layer structure, comprising an inner band portion 307 and an outer band portion 309. The inner band portion 307 and the outer band portion 309 are separably integrated together by a hook-and-loop fastener (not shown) or the like.

As illustrated in FIG. 18B, the inner band portion 307 is wider than the outer band portion 309 throughout.

The inner band portion 307 (or its material) is superior to the outer band portion 309 (or its material) in one or more of the following characteristics, i.e., flexibility, contractility, water absorbing property, air permeability, the favorableness of contact, and light blocking effect. For example, the inner band portion 307 is formed of pile fabric material (like supporters) or casual rubber whose surface is covered with cloth material such as towel cloth, or the like. The outer band portion 309 is formed of harder rubber material, resin, or the like.

In this embodiment, the inner band portion 307 excellent in flexibility or contractility offers the enhanced favorableness of contact with an arm or the like owing to the above-mentioned constitution. Pressing force is prevented from being excessively applied to part of an arm or the like, and thus excellent sense of use is obtained. The inner band portion is excellent in water absorbing property or air permeability, which also provides excellent sense of use.

Further, the inner band portion 307 is excellent in the favorableness of contact, large in width, high in light blocking effect, and excellent in other like properties. Thus, external light is less prone to stream into the gap between the pulse wave sensor main unit 303 and the inner band portion 307, and this brings the advantage of the enhanced measurement accuracy.

Furthermore, it is possible to wash only the inner band portion 307, which is brought into contact with an arm and is thus prone to be soiled, by separating the inner band portion 307 and the outer band portion 309 from each other.

### Sixteenth Embodiment

Description will be given to a pulse wave sensor in a sixteenth embodiment. The same description as of the first embodiment will be omitted.

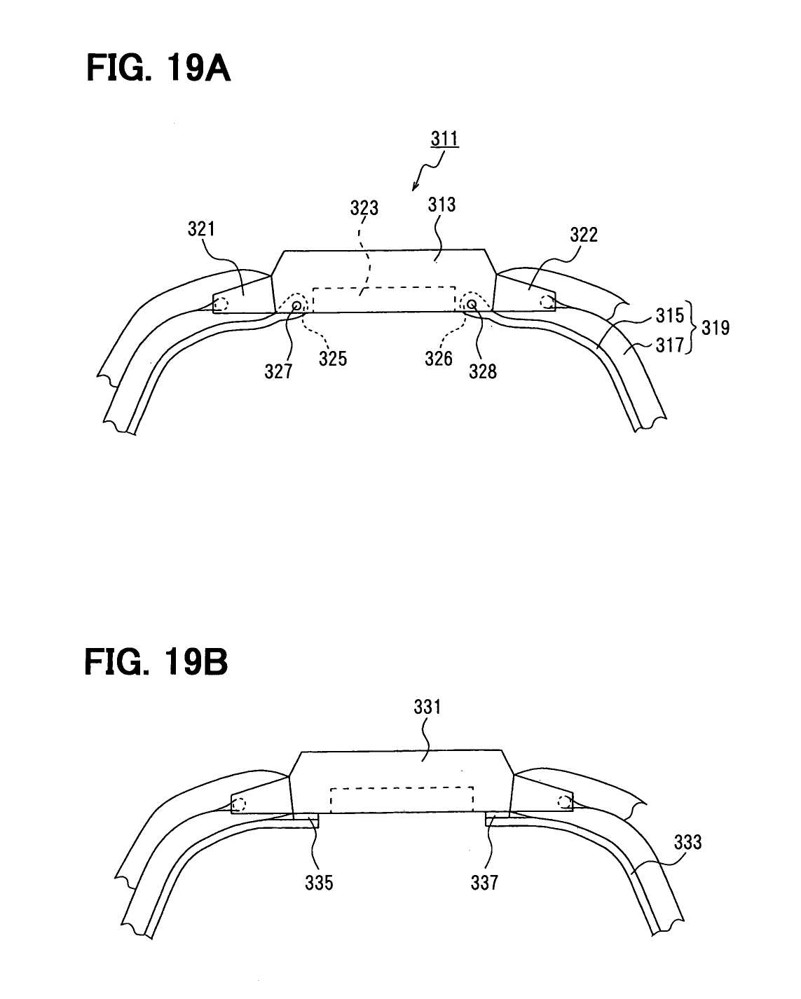

As illustrated in FIG. 19A, the pulse wave sensor 311 in this embodiment comprises a plate-like pulse wave sensor main unit 313 and a band 319 consisting of an inner band portion 315 and an outer band portion 317, like that in the fifteenth embodiment.

In this embodiment, especially, the attachment positions of the inner band portion 315 and the outer band portion 317 are different from those in the fifteenth embodiment.

More specific description will be given. The outer band portion 317 is attached to sensor anchoring portions 321 and 322 extended from the pulse wave sensor main unit 313 as in the fifteenth embodiment. Meanwhile, the inner band portion 315 is fixed on the inner surface of the pulse wave sensor main unit 313 on both sides of the measurement arrangement 323.

More specific description will be given. Recesses 325 and 326 extended in the direction of the depth of the figure are formed in the inner surface of the pulse wave sensor main unit 313. Fixing rods 327 and 328 that can be detached (by contracting them in the axial direction) are installed in the recesses 325 and 326. The ends of the inner band portion 315 are wrapped around the fixing rods 327 and 328, and the inner band portion 315 is thereby fixed. Therefore, the inner band portion 315 can be removed from the pulse wave sensor main unit 313 by contracting the fixing rods 327 and 328.

With this constitution, the inner band portion 315 can be brought into tighter contact with an arm or the like. This brings the advantages that the pulse wave sensor 311 is less prone to be displaced and the measurement accuracy is further enhanced.

Another constitution is illustrated in FIG. 19B. In this constitution, the pulse wave sensor main unit 331 and the inner band portion 333 are detachably attached to each other by hook-and-loop fasteners 335 and 337.

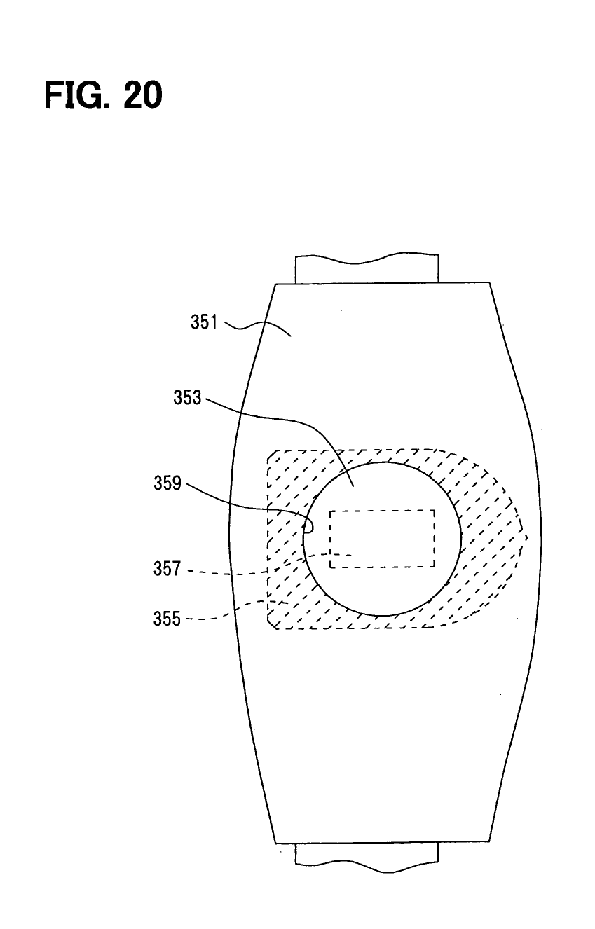

When the inner band portion 351 is attached to the pulse wave sensor main unit 353 by the hook-and-loop fastener 355 or the like, the constitution illustrated in FIG. 20 can be adopted. In this constitution, an opening 359 is formed in the inner band portion 351 so that the measurement arrangement 357 is encircled with the outline of the opening. (That is, the opening 359 is formed by hollowing the inner band portion in the position corresponding to the measurement arrangement 357.) Thus, the light blocking effect is further enhanced.

### Seventeenth Embodiment

Description will be given to a pulse wave sensor in a seventeenth embodiment. The same description as of the first embodiment will be omitted.

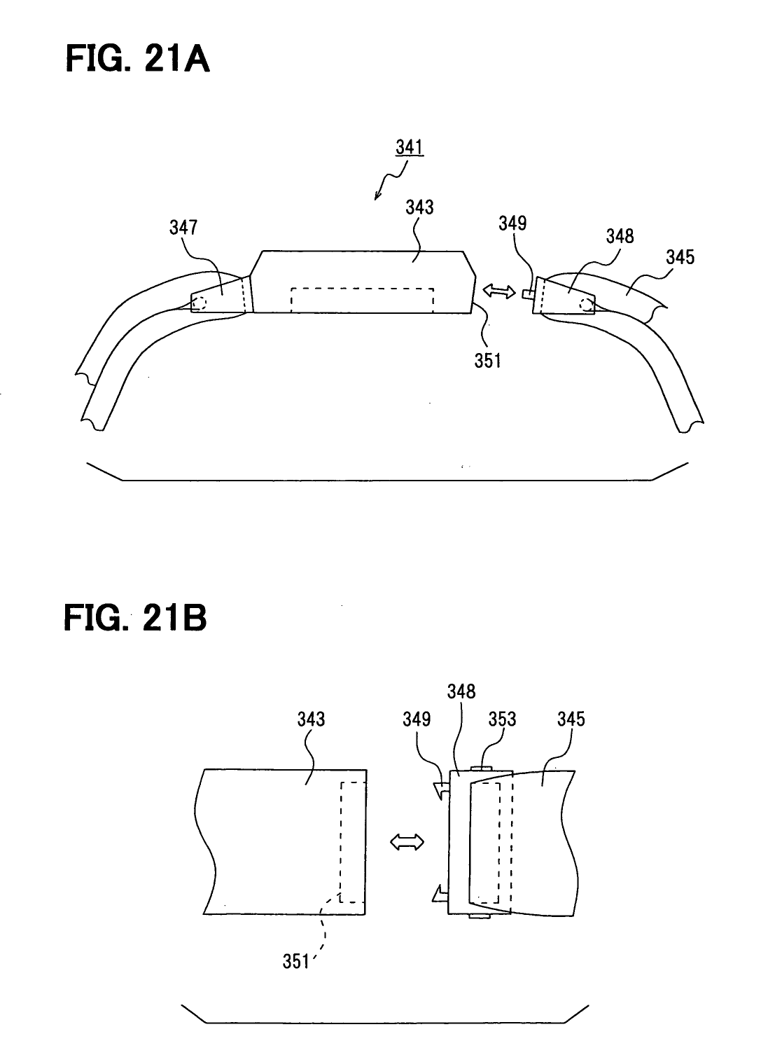

As illustrated in FIG. 21A, the pulse wave sensor 341 in this embodiment comprises a plate-like pulse wave sensor main unit 343 and an elastic long strip-shaped band 345, like that in the first embodiment.

In this embodiment, especially, the pulse wave sensor main unit 343 and band anchoring portions 347 and 348 to which the band 345 is attached are detachably constructed.

More specific description will be given. As illustrated in FIG. 21B, the band anchoring portions 347 and 348 are provided with a pair of hook-shaped anchoring portion-side engaging portions 349; the pulse wave sensor main unit 343 is provided with recessed main unit-side engaging portions 351. The anchoring portion-side engaging portions 349 and the main unit-side engaging portions 351 are detachably engaged with each other. Thus, the pulse wave sensor main unit 343 and the band anchoring portions 347 and 348 are integrated with each other.

Pairs of the anchoring portion-side engaging portions 349 are energized outward. When the anchoring portion-side engaging portions 349 are pushed into the main unit-side engaging portions 351, they are engaged with internal rods (not shown). When push buttons 353 are pressed, the anchoring portion-side engaging portions 349 are energized inward, and disengaged from the main unit-side engaging portions 351.

In this embodiment, as mentioned above, the band anchoring portions 347 and 348 are not integrally fixed on the pulse wave sensor main unit 343, and they are detachable from the pulse wave sensor main unit 343. When the pulse wave sensor 341 is attached around an arm or the like, the band anchoring portions 347 and 348 only have to be removed from the pulse wave sensor main unit 343. Thus, the pulse wave sensor 341 can be very easily attached and detached.

The constitution in which only one of the band anchoring portions 347 and 348 is detachable may be adopted.

The present invention is not limited to the above-mentioned embodiments, and various modifications are obviously possible without departing from the scope of the present invention.

For example, a pressure sensor or the like may be disposed to the inner side of the sensor main unit or of the band. Thus, when the pressing force is appropriate or inappropriate, a person as the subject of measurement can be informed of that.

The band insertion holes may be in such shape that they are partly notched and open to the outside.

The present invention can be summarized as follows.

(1) According to one aspect of the present invention, as illustrated in FIG. 1, the flexible elastic band of, for example, rubber is deformed and passed through the band insertion holes of the band anchoring sections. Thus, the band is anchored in the band insertion holes (and thus at the band anchoring sections) by elastic force arising from the deformation thereof to limit displacement of the band. More specifically, the portions of the band passed through the band insertion holes are compressed and are recovered from deformed state after they exit the band insertion holes. Thus, the band is anchored at the band anchoring sections so that it will not be displaced unless a large external force, which is greater than a predetermined value and causes displacement of the engaging positions of the band relative to the insertion holes, is applied to the band.

As a result, the measurement arrangement is pressed toward the living body by elastic force of the band, and the sensor main unit is attached to the measurement region of the living body. Thus, accurate measurement becomes possible.

Therefore, the body condition measuring device of the present invention is simple in its structure as compared with the conventional wrist watch-type sensors that are fixed by inserting a pin into any of a plurality of fixing holes in a band. Further, the length of the band of the body condition measuring device of the present invention is linearly adjustable, and it is easily adjustable. Therefore, accurate measurement is possible.

Specifically, according to this aspect of the present invention, the length of the band can be adjusted to adjust the fastening force just by passing the band through the band insertion holes and pulling it. When pulling is stopped, the band is brought into anchored state there (by elastic force arising from deformation of the band itself). Thus, the body condition measuring device is easy to use and convenient.

Side ends of the sensor main unit are opposed to each other in a plane of the measurement arrangement of the sensor main unit. In the case of the plate-like sensor main unit, the side ends of the sensor main unit are opposed to each other in a direction perpendicular to the plate thickness direction of the plate-like sensor main unit.

Possible materials for the band include: rubber materials woven of rubber string, casual rubber whose surface is covered with cloth material, pile fabric materials (like supporters) whose surface is covered with towel cloth, and the like. Definitions of the degree of deformation include deformation within the range of 20% or more in one direction (e.g. the direction of the thickness of the band) and 50% or more in the direction of length. (The foregoing is the same with the following claims.)

(2) According to another aspect of the present invention, a plate-like sensor main unit is provided with a band anchoring portion at both ends of the main unit in the direction of wrapping the band, as illustrated in FIGS. 9A and 9B. In this case, the band can be made annular by passing both the ends of the band through the band insertion holes in the band anchoring portions positioned on the left and right in the figures. The user inserts his/her attachment region such as his/hear wrist into the ring of the band, and tightens the band. Thus, the sensor main unit (and thus the body condition measuring device) can be attached to the attachment region with ease.

(3) According to another aspect of the present invention, as illustrated in FIG. 12, the band anchoring portions project obliquely from the ends of the sensor main unit outward and downward toward the attachment region of the body as viewed sideways. Specifically, the band anchoring portions are protruded in a direction between the direction in which the plate-like sensor main unit extends and the place in which the attachment region is to be positioned. Therefore, the sensor main unit and the band anchoring portions form substantially L shape, as viewed sideways. This brings the advantage that the sensor main unit and the band are easily brought into tight contact with an attachment region such as a wrist.

(4) According to another aspect of the present invention, as illustrated in FIGS. 15A to 15C, the band anchoring portions can be opened at the band insertion holes and can be detached from the sensor main unit (the rest of the sensor main unit). Therefore, a band of bigger dimensions can be easily placed in the band insertion holes.

(5) According to another aspect of the present invention, as illustrated in FIGS. 15A to 15C, the band anchoring portions can be moved relative to the sensor main unit (the rest of the sensor main unit) by rotation or linear movement. Therefore, a band of bigger dimensions can be easily placed in the band insertion holes.

(6) According to another aspect of the present invention, the one of the outer dimension or the cross-sectional area of the band is larger than the corresponding one of the inner dimension and the cross-sectional area of the band insertion hole as illustrated in FIG. 1. Therefore, when the band is passed through the band insertion holes, the band is deformed. Thus, the band is anchored at the band anchoring portions and fixed there by its elastic force arising.

In case of a long strip-shaped band, the above-mentioned inner dimension and outer dimension may be the dimension in the thickness direction of the band or the dimension in the width direction of the band.

Examples of comparisons of dimensions and cross-sectional area include the following cases. The inner dimension of the band insertion hole may be within the range of 40 to 90% of the outer dimension of the band. Furthermore, the cross-sectional area of the band insertion hole may be within the range of 40 to 90% of the cross-sectional area of the band.

(7) According to another aspect of the present invention, when the body condition measuring device is attached to an attachment region such as the wrist, as illustrated in FIG. 1, the band is stretched in accordance with the dimensions of the attachment region. Thus, the band presses the sensor main unit (and thus the measurement arrangement) against the measurement region by its pressing force. Therefore, the inner diameter of the ring (the annular portion) of the band is increased. When the body condition measuring device is removed from the attachment region, the band is contracted by its own elastic force. As a result, the inner diameter of the ring (the annular portion) of the band is reduced.

(8) According to another aspect of the present invention, as illustrated in FIG. 2, the band is passed through the band insertion holes from the inner side (measurement arrangement side: living body side) to the outer side, and the ends of the band are disposed to the outer side. Therefore, the band is prone to be brought into tight contact with the attachment region, and the fastening force of the band can be adjusted with ease.

(9) According to another aspect of the present invention, when the body condition measuring device is not attached to an attachment region such as a wrist, as illustrated in FIG. 2, the band is contracted by its own elastic force. However, its portions in the band insertion holes are anchored at the band anchoring portions and prevented from being displaced by elastic force arising from deformation of the band. The engaging position of the band relative to the band insertion hole of the band anchoring portion is unshiftable unless an external force greater than a predetermined value is applied to the band.

More specifically, even when the band is pulled with such force as to be exerted when the body condition measuring device is normally attached, the band is not displaced from the band insertion holes. Therefore, when the body condition measuring device is not attached and is left in a natural state, for example, the anchoring positions of the band are not shifted. When a user wears the measuring device next time, he/she can attach it under the same conditions (e.g. pressing force) as he/she previously wore it. Thus, the constant measurement accuracy can be maintained.

(10) According to another aspect of the present invention, as illustrated in FIG. 11, the surface of the sensor main unit on the measurement arrangement side is, for example, concavely curved in correspondence with the shape of the surface of an attachment region such as a wrist. This brings the following advantage. That is, when the body condition measuring device is attached to an attachment region, such as the wrist, the sensor main unit is favorably brought into tight contact with the attachment region.

(11) According to another aspect of the present invention, as illustrated in FIG. 11, the surface of the sensor main unit on the measurement arrangement side is, for example, structurally concavely curved in the predetermined single direction in accordance with the surface shape of the attachment region. An example of the one predetermined direction is the circumferential direction of the wrist (the direction of wrapping the band). This brings the advantage that the sensor main unit is favorably brought into tight contact with the attachment region.

(12) According to another aspect of the present invention, as illustrated in FIGS. 6A and 6B, the band is passed through the band insertion holes, and the both the ends of the band are so big-sized that they cannot be passed through the band insertion holes. Thus, even when the band is pulled hard, the band does not come off the band insertion holes.

One end of the band may be so big-sized that it cannot be passed through the band insertion hole. In this case, the band does not come off the band insertion hole even when the other end of the band is pulled hard.

As constitutions for preventing the band end from being passed through the band insertion hole, for example, the following methods can be adopted. That is, it could be a method in which the dimensions such as thickness and width of the band are made so big that the band cannot be passed through the band insertion holes. Alternatively, it could be a method in which after the band is passed through the band insertion holes, for example, a hard member bigger in size than the band insertion holes is installed at one or both ends of the band.

(13) According to another aspect of the present invention, the band itself is anchored in the band insertion holes by elastic force a rising from deformation of the band itself. As illustrated in FIG. 7 as an example, therefore, the anchoring positions of the band can be adjusted by pulling the band with force greater than this anchoring force arising from elasticity. One end of the band can be fixed by bonding, sewing, use of a fastener, or the like, as illustrated in FIG. 7 as an example. In this case, even when the other end of the band is pulled, the band does not come off.

When an annular band is attached around a wrist or the like, for example, it is undesirable that the anchoring positions (the engaging positions) are shifted only by widening the ring of the band. Therefore, it is preferable that elastic force should be appropriately adjusted so that the anchoring positions will not be shifted under the force big to such an extent, exerted when the body condition measuring device is attached.

(14) According to another aspect of the present invention, as illustrated in FIG. 8, when a user wears the body condition measuring device around his/her wrist, for example, the free end of the band comes to the user side (the human body side of the wrist, i.e., the thumb-side of the wrist), so that the engaging position of the band is adjustable on the thumb-side of the wrist in the installed state of the band relative to the wrist. Thus, the anchoring positions of the band can be adjusted with ease.

The orientation of the sensor main unit can be adjusted according to, for example, the orientation of the display portion of the sensor main unit or marking. Therefore, the side on which the length of the band is adjusted can be correspondingly set.

(15) According to another aspect of the present invention, the shape illustrated in FIG. 1 can be adopted. In FIG. 1, the sensor main unit is asymmetrical with respect to the left-right direction, i.e., the first direction. This prevents the body condition measuring device from being attached with the sensor main unit upside down. Therefore, consistent measuring conditions can be always maintained, and this brings the advantage of enhanced measurement accuracy. Alternatively, the sensor main unit may be asymmetrical with respect to a perpendicular direction (the second direction), which is perpendicular to the left-right direction, i.e., the first direction.

(16) According to another aspect of the present invention, as illustrated in FIGS. 9A and 9B, one or both ends of the band passed through the band insertion holes are secured on the surface of the band or the like by sewing, bonding, a hook-and-loop fastener, or the like. Thus, even when the band is pulled hard, the band is prevented from coming off the band insertion holes.

(17) According to another aspect of the present invention, as illustrated in FIG. 9C, the free end of the band is passed through the ring member and is thereby secured. This prevents the free end of band from jumping and obstructively behaving.

(18) According to another aspect of the present invention, as illustrated in FIG. 10A, the band covers the outer side of the sensor main unit (the side opposite the measurement arrangement), and is secured on the surface of the band or the like positioned on the opposite side. This brings the advantage that the sensor main unit is firmly secured.

(19) According to another aspect of the present invention, as illustrated in FIG. 10B, the band is passed through the hollow portion, and secured on the surface of the band or the like positioned on the opposite side. Therefore, the sensor main unit can be firmly secured. Furthermore, when the sensor main unit is provided on its surface with a display portion, the user can view the indication on the display.

(20) According to another aspect of the present invention, as illustrated in FIG. 1, the sensor main unit is provided at its side with the operating portion including operating switches or the like for operating the body condition measuring device, like a wrist watch. Therefore, the measuring device can be operated with ease.

(21) According to another aspect of the present invention, as illustrated in FIG. 16A, the operating portion including operating switches or the like is provided in the recess. This brings the advantage that someone or something is less prone to be caught on the operating switches or the like.

(22) According to another aspect of the present invention, as illustrated in FIG. 2, the band is a single elongated strip-shaped member. Therefore, the band is simple in structure, and is easy to handle when it is attached to the sensor main unit.

(23) According to another aspect of the present invention, the band is not constant in width along its length but is wider on the side opposite from the measurement arrangement.

The constitution illustrated in FIG. 17A as an example can be adopted. Thus, the body condition measuring device becomes less prone to be displaced, and the measurement accuracy is enhanced.

(24) According to another aspect of the present invention, an auxiliary pad wider than the band is provided on the band on the side opposite from the measurement arrangement.

The constitution illustrated in FIG. 17B as an example can be adopted. Thus, the body condition measuring device becomes less prone to be displaced, and the measurement accuracy is enhanced.

(25) According to another aspect of the present invention, as illustrated in FIG. 18A as an example, the band includes an inner band portion and an outer band portion, which are overlapped with each other. The inner band portion is brought into contact with the wrist or the like, and the outer band portion is positioned outside the inner band portion. The material of the inner band portion and the material of the outer band portion differ from one another. Therefore, materials most suitable for the measuring device for measuring the conditions of the living body can be selected and used. For example, a material excellent in flexibility can be adopted for the inner band portion, and a slightly harder material can be adopted for the outer band portion. Thus, the inner band portion is brought into tight contact with a wrist or the like, and the outer band portion can firmly hold the sensor main unit, inner band portion, and the like.

(26) According to another aspect of the present invention, the inner band portion has greater flexibility in comparison to the outer band portion. Therefore, the inner band portion is brought into tight contact with the surface of the wrist or the like, and thus the body condition measuring device is less prone to be displaced. In the case where measurement is made by light to collect living body information, external light is less prone to stream into the gap between the inner band portion and the wrist or the like. Thus, the measurement accuracy is enhanced. Since the inner band portion is flexible, pressing force is prevented from being excessively applied only to part of the wrist or the like, and this provides excellent sense of use. Since the outer band portion is harder than the inner band portion, meanwhile, it is capable of firmly holding the measuring device.

(27) According to another aspect of the present invention, the inner band portion has greater stretchability in comparison to the outer band portion.

(28) According to another aspect of the present invention, the inner band portion has greater water absorbability in comparison to the outer band portion.

In this aspect of the present invention, the inner band portion is excellent in water absorbing property, and this brings about the effect of preventing degradation in sense of use even when the user becomes sweaty.

(29) According to another aspect of the present invention, the inner band portion has greater air permeability in comparison to the outer band portion.

In this aspect of the present invention, the inner band portion is excellent in air permeability, and this brings about the effect of preventing degradation in sense of use even when the user becomes sweaty.

(30) According to another aspect of the present invention, the inner band portion has a greater degree of adhesion relative to the living body in comparison to the outer band portion. More specifically, the inner band portion can fit more tightly relative to the living body in comparison to the outer band portion.

In this aspect of the present invention, the inner band portion is superior to the outer band portion in the favorableness of contact, and this brings about the same effect as the aspect of the invention described in the above section (26).

(31) According to another aspect of the present invention, the inner band portion exhibits higher light blocking effect in comparison to the outer band portion.

In this aspect of the present invention, the inner band portion is superior to the outer band portion in light blocking effect. In the case where measurement is made by light to collect living body information, external light is less prone to stream into the gap between the inner band portion and the wrist or the like. Thus, the measurement accuracy is enhanced.

(32) According to another aspect of the present invention, a majority of the inner band portion (a region of the inner band portion that is equal to greater than one half of the inner band portion) has a width greater than that of a majority of the outer band portion.

As illustrated in FIG. 18B as an example, in this aspect of the present invention, the width of the inner band portion is greater than that of the outer band portion as a whole. Therefore, the band is less prone to be displaced, and is excellent in light blocking effect. In case measurement is made by light to collect living body information, external light is less prone to stream into the gap between the inner band portion and the wrist or the like. Thus, the measurement accuracy is enhanced.

(33) According to another aspect of the present invention, the inner band portion and the outer band portion are separable from each other.

In this aspect of the present invention, the inner band portion (which is brought into contact with the wrist or the like and is thus prone to be soiled) and the outer band portion are separable from each other. The inner band portion and the outer band portion are separably joined together using, for example, a hook-and-loop fastener. Therefore, only the separated inner band portion can be washed.

(34) According to another aspect of the present invention, the inner band portion and the outer band portion are separately fixed to the sensor main unit.

As illustrated in FIGS. 19A and 19B, this aspect of the present invention is so constructed that the inner band portion and the outer band portion are attached to the sensor main unit in different positions. Therefore, for example, the inner band portion can be attached to the rear side (the side on which the wrist or the like is positioned) of the sensor main unit so that the wrist or the like is sufficiently covered with the inner band portion; the outer band portion can be attached to side ends of the sensor main unit. Thus, the favorableness of contact of the inner band portion can be enhanced.

(35) According to another aspect of the present invention, a detecting arrangement and a notifying arrangement are additionally included in the measuring device. The detecting arrangement detects the state of attachment of the measurement arrangement to the human body. When the state of attachment of the measurement arrangement is detected by the detecting arrangement, the notifying arrangement notifies of the result of detection. The detecting arrangement may be integrally provided in the measurement arrangement of any one or more of the above embodiments, and the notifying arrangement may be integrally provided in the display portion (the monitor) of any one or more of the above embodiments.

Thus, it can be learnt whether the measurement arrangement is properly attached or not, and the state of attachment can be grasped with accuracy.

(36) According to another aspect of the present invention, the pulse waves in the living body are detected by the measurement arrangement.

FIGS. 5A and 5B illustrate an example of a body condition measuring device according to this aspect of the present invention.

(37) According to another aspect of the present invention, the measurement arrangement is provided with a light emitter and a light receiver. The light emitter projects light to a measurement region of the living body. The light receiver receives the projected light reflected by the living body, and generates a living body information signal corresponding to the amount of the received light.

FIGS. 5A and 5B illustrate an example of a measurement arrangement according to this aspect of the present invention. In this aspect of the present invention, use of the above-mentioned band brings the measurement arrangement into tight contact with a measurement region, and makes displacement less prone to occur. Therefore, this aspect of the present invention is most suitable for instruments that carry out such optical measurement.

(38) According to another aspect of the present invention, as illustrated in FIG. 4, the band anchoring portions are provided at side ends of the sensor main unit. For example, the band anchoring portions are provided so that they are protruded outward.

In this case, conventional technologies are prone to pose a problem. Gaps are prone to be produced between the recesses formed by the inner surface of the band and the inner surface of the sensor main unit, and the attachment region of the living body. (The inner surfaces of the band and the sensor main unit are both their surfaces positioned on the attachment region side.) This can lead to displacement of the sensor main unit in the direction of wrapping the band.

As mentioned above, this aspect of the present invention is so constructed that the band formed of flexibly deformable elastic material is passed through the band insertion holes. Therefore, gaps are not produced between the inner surface of the band and the inner surface of the sensor main unit, and the attachment region, and the sensor main unit is brought into tight contact with the attachment region. Consequently, the sensor main unit is less prone to be displaced, and the following advantages are brought. That is, noise signals are less prone to be produced, and the measurement accuracy is enhanced.

As mentioned above, it is preferable that by elastic force arising from deformation of the band itself, the band should anchor the band itself in the band insertion holes and further press the measurement arrangement against the living body.

(39) According to another aspect of the present invention, as illustrated in FIGS. 21A and 21B, the band anchoring portions are not integrally fixed on the sensor main unit but can be detached from the sensor main unit (the rest of the sensor main unit). Therefore, when the body condition measuring device is attached to the wrist or the like, the band anchoring portions can be removed from the sensor main unit. This significantly facilitates attachment and detachment of the body condition measuring device.

(40) According to another aspect of the present invention, the sensor main unit is provided with the band insertion holes, as illustrated in FIG. 13. Therefore, the following advantage is brought. That is, when anything hits the sensor main unit, the sensor main unit is less prone to be displaced as compared with cases where the band anchoring portions are protruded from the sensor main unit.

(41) According to another aspect of the present invention, as illustrated in FIGS. 14A to 14C, a component(s), such as a measurement arrangement, required for measuring of the condition of the living body is provided to the band. This brings about the following effects. That is, the favorable contact is obtained between the component(s) and the attachment region, and the body condition measuring device can be made compact.

(42) According to another aspect of the present invention, as illustrated in FIGS. 14A to 14C, the component(s) required for measuring of the condition of the living body is disposed on (for example, built in) the band. The component(s) may include at least one of the measurement arrangement, a display portion, a battery and an operating portion. The display portion, which serves as the component, is a device that indicates the result of measurement and the like by liquid crystal or the like.

(43) According to another aspect of the present invention, the measurement arrangement is provided separately from the band, and the component(s) provided to the band is electrically connected to the measurement arrangement through a connecting structure.

When the band is provided with the display portion as illustrated in FIG. 14A, the display portion can be connected to the measurement arrangement (and thus the sensor main unit) by, for example, button-like connecting structures.

(44) According to another aspect of the present invention, as illustrated in FIG. 16A, a sensor main unit is fixed to the inner side of, for example, the integral annular band using, for example, a hook-and-loop fastener. Thus, the body condition measuring device can be constructed with ease.

(45) According to another aspect of the present invention, the body condition measuring device is provided with a fixing structure (e.g. hook-and-loop fastener) that is provided on the surface of the sensor main unit on one side in the direction of plate thickness, and fixes the sensor main unit itself on the band.

FIG. 16B that illustrates this aspect of the present invention as an example shows an example of the structure of the sensor main unit used in the aspect of the invention described in claim 28.

Additional advantages and modifications will readily occur to those skilled in the art. The invention in its broader terms is therefore not limited to the specific details, representative apparatus, and illustrative examples shown and described.

1. A body condition measuring device comprising:
a sensor main unit having a measurement arrangement, wherein the measurement arrangement contacts a living body and measures a condition of the living body; and
a band that is wrapped around an attachment region of the living body and attaches the sensor main unit to the attachment region of the living body, wherein:
the sensor main unit has at least one band anchoring portion, each of which is positioned at a corresponding one of opposed ends of the sensor main unit and has a band insertion hole for receiving the band therethrough;
the band is substantially made of a flexibly deformable elastic material and is anchored in the band insertion hole of each of the at least one band anchoring portion due to an elastic force generated upon deformation of the band; and
the band urges the measurement arrangement against the living body due to the elastic force generated upon deformation of the band.

2. The body condition measuring device according to claim 1, wherein:
the at least one band anchoring portion includes two band anchoring portions, which are positioned to the opposed ends, respectively, of the sensor main unit; and
longitudinal ends of the band are anchored to the two band anchoring portions, respectively, so that the band is configured to be annular.

3. The body condition measuring device according to claim 1, wherein each of the at least one band anchoring portion projects obliquely from the corresponding one of the opposed ends of the sensor main unit toward the attachment region of the living body.

4. The body condition measuring device according to claim 1, wherein each of the at least one band anchoring portion has a detachable structure, which opens at the band insertion hole thereof relative to the rest of the sensor main unit.

5. The body condition measuring device according to claim 1, wherein each of the at least one band anchoring portion has a movable structure, which opens at the band insertion hole thereof relative to the rest of the sensor main unit by making one of rotational movement and linear movement relative to the rest of the sensor main unit.

6. The body condition measuring device according to claim 1, wherein one of an outer dimension and a cross-sectional area of the band is larger than a corresponding one of an inner dimension and a cross-sectional area of the band insertion hole of each of the at least one band anchoring portion.

7. The body condition measuring device according to claim 1, wherein a diameter of an annular portion of the band, which surrounds the attachment region of the living body, varies between an installed state of the band relative to the attachment region and an uninstalled state of the band relative to the attachment region.

8. The body condition measuring device according to claim 1, wherein the band is inserted through the band insertion hole of each of the at least one band anchoring portion from an inner side of the band anchoring portion where the measurement arrangement is provided to an outer side of the band anchoring portion, so that each corresponding one of longitudinal ends of the band is disposed at the outer side of the corresponding band anchoring portion.

9. The body condition measuring device according to claim 1, wherein:
the band is anchored in the band insertion hole of each of the at least one band anchoring portion in an uninstalled state of the band relative to the attachment region of the living body due to elasticity of the bald; and
an engaging position of the band relative to the band insertion hole of each of the at least one band anchoring portion is unshiftable unless an external force greater than a predetermined value is applied to the band.

10. The body condition measuring device according to claim 1, wherein a surface of the measurement arrangement of the sensor main unit is curved.

11. The body condition measuring device according to claim 10, wherein the curved surface of the measurement arrangement of the sensor main unit is curved in a predetermined single direction in conformity with a shape of a surface of the attachment region of the living body.

12. The body condition measuring device according to claim 1, wherein at least one of longitudinal ends of the band is prevented from passing through the band insertion hole of each of the at least one band anchoring portion.

13. The body condition measuring device according to claim 1, wherein an engaging position of the band relative to the band insertion hole of the at least one band anchoring portion is adjustable in at least one of longitudinal ends of the band.

14. The body condition measuring device according to claim 13, wherein:
the attachment region of the living body is a wrist of the living body; and
the engaging position of the band is adjustable on a thumb-side of the wrist of the living body in an installed state of the band relative to the wrist.

15. The body condition measuring device according to claim 1, wherein:
the sensor main unit is asymmetrical in one of a first direction and a second direction;
the first direction extends in a left-right direction of a plane of the sensor main unit; and
the second direction is perpendicular to the first direction in the plane of the sensor main unit.

16. The body condition measuring device according to claim 1, wherein at least one of longitudinal ends of the band is securable.

17. The body condition measuring device according to claim 1, wherein a longitudinal free end of the band is received through and is secured to a ring member, which is fitted around the band.

18. The body condition measuring device according to claim 1, wherein the band is secured in such a manner that the band covers an outer side of the sensor main unit.

19. The body condition measuring device according to claim 1, wherein:
the sensor main unit includes a hollow portion, which penetrates through the sensor main unit and thereby connects between the opposed ends of the sensor main unit; and
the band passes through the hollow portion of the sensor main unit and is secured.

20. The body condition measuring device according to claim 1, wherein a side of the sensor main unit includes an operating portion for operating the body condition measuring device.

21. The body condition measuring device according to claim 20, wherein:
the side of the sensor main unit includes a recess; and
the operating portion is arranged in the recess of the side of the sensor main unit.

22. The body condition measuring device according to claim 1, wherein the band is a single elongated strip-shaped member.

23. The body condition measuring device according to claim 1, wherein:
a width of the band is not constant along a length of the band; and
an opposite portion of the band, which is opposite from the measurement arrangement, is wider than an adjacent portion of the band, which is adjacent to the measurement arrangement.

24. The body condition measuring device according to claim 1, wherein an opposite portion of the band, which is opposite from the measurement arrangement, is provided with an auxiliary pad, which has a width that is greater than that of the opposite portion of the band.

25. The body condition measuring device according to claim 1, wherein:
the band includes an inner band portion and an outer band portion, which are overlapped with each other; and
a material of the inner band portion and a material of the outer band portion differ from one another.

26. The body condition measuring device according to claim 25, wherein the inner band portion has greater flexibility in comparison to the outer band portion.

27. The body condition measuring device according to claim 25, wherein the inner band portion has greater stretchability in comparison to the outer band portion.

28. The body condition measuring device according to claim 25, wherein the inner band portion has greater water absorbability in comparison to the outer band portion.

29. The body condition measuring device according to claim 25, wherein the inner band portion has greater air permeability in comparison to the outer band portion.

30. The body condition measuring device according to claim 25, wherein the inner band portion has a greater degree of adhesion relative to the living body in comparison to the outer band portion.

31. The body condition measuring device according to claim 25, wherein the inner band portion exhibits higher light blocking effect in comparison to the outer band portion.

32. The body condition measuring device according to claim 25, wherein a majority of the inner band portion has a width greater than that of a majority of the outer band portion.

33. The body condition measuring device according to claim 25, wherein the inner band portion and the outer band portion are separable from each other.

34. The body condition measuring device according to claim 25, wherein the inner band portion and the outer band portion are separately fixed to the sensor main unit.

35. The body condition measuring device according to claim 1, further comprising:
a detecting arrangement that detects a state of attachment of the measurement arrangement relative to the attachment region of the living body; and
a notifying arrangement that notifies a detected result of the detecting arrangement when the state of the attachment of the measurement arrangement is detected by the detecting arrangement.

36. The body condition measuring device according to claim 1, wherein the measurement arrangement detects pulse waves of the living body.

37. The body condition measuring device according to claim 36, wherein the measurement arrangement includes:
a light emitter that projects light to a measurement region of the living body; and
a light receiver that receives the projected light reflected by the living body and generates a living body information signal, which corresponds to an amount of the light received by the light receiver.

38. A body condition measuring device comprising:
a sensor main unit having a measurement arrangement, wherein the measurement arrangement contacts a living body and measures a condition of the living body; and
a band, that is wrapped around an attachment region of the living body and attaches the sensor main unit to the attachment region of the living body, wherein:
the sensor main unit has at least one band anchoring portion, each of which is positioned at a corresponding one of opposed ends of the sensor main unit and has a band insertion hole for receiving the band therethrough;
the band is substantially made of a flexibly deformable elastic material;
an inner surface of the band and an inner surface of the sensor main unit form a concave section, which tightly contacts the attachment region of the living body.

39. A body condition measuring device comprising:
a sensor main unit having a measurement arrangement, wherein the measurement arrangement contacts a living body and measures a condition of the living body; and
a band that is wrapped around an attachment region of the living body and attaches the sensor main unit to the attachment region of the living body, wherein:
the sensor main unit has at least one band anchoring portion, each of which is detachable relative to the rest of the sensor main unit and has a band insertion hole for receiving the band therethrough;
the band is substantially made of a flexibly deformable elastic material; and
the band urges the measurement arrangement against the living body due to an elastic force generated upon deformation of the band in a state where the at least one band anchoring portion is installed to the rest of the sensor main unit.

40. A body condition measuring device comprising:
a sensor main unit having a measurement arrangement, wherein the measurement arrangement contacts a living body and measures a condition of the living body; and
a band that is wrapped around an attachment region of the living body and attaches the sensor main unit to the attachment region of the living body, wherein:
the sensor main unit has at least one band insertion hole for receiving the band therethrough;
the band is substantially made of a flexibly deformable elastic material and is anchored in the band insertion hole of each of the at least one band anchoring portion due to an elastic force generated upon deformation of the band; and
the band urges the measurement arrangement against the living body due to the elastic force generated upon deformation of the band.

41. A body condition measuring device comprising:
a measurement arrangement that contacts a living body and measures a condition of the living body; and
a band that is wrapped around an attachment region of the living body and urges the measurement arrangement against the attachment region of the living body, wherein at least one component required for measuring of the condition of the living body is provided to the band.

42. The body condition measuring device according to claim 41, wherein the at least one component includes at least one of the measurement arrangement, a display portion, a battery and an operating portion.

43. The body condition measuring device according to claim 41, wherein the measurement arrangement is provided separately from the band, and the at least one component provided to the band is electrically connected to the measurement arrangement through a connecting structure.

44. A body condition measuring device comprising:
a sensor main unit having a measurement arrangement, wherein the measurement arrangement contacts a living body and measures a condition of the living body; and
a band that is wrapped around an attachment region of the living body and urges the measurement arrangement against the attachment region of the living body, wherein the sensor main unit is secured to an inner side of the band through a fixing structure.

45. A body condition measuring device comprising:
a planar sensor main unit, which receives a measurement arrangement, wherein the measurement arrangement contacts a living body and measures a condition of the living body;
a band that is substantially made of a flexible deformable elastic material and is wrapped around an attachment region of the living body, wherein the band urges the measurement arrangement against the attachment region of the living body; and
a fixing structure that is provided in one of opposed surfaces of the sensor main unit, which are opposed to each other in a thickness direction of the sensor main unit, wherein the fixing structure fixes the sensor main unit to the band.
